(19) United States
(12) Patent Application Publication
(10) Pub. No.: US 2023/0089175 A1
(43) Pub. Date: Mar. 23, 2023

(54) Rice event GSX2-55

(71) Applicant: CONNECTICUT BIOTECHNOLOGY CORPORATION, Hamden, CT (US)

(72) Inventors: Ning WEI, Hamden, CT (US); Xingwang DENG, Hamden, CT (US)

(21) Appl. No.: 17/798,892

(22) PCT Filed: Apr. 16, 2020
(86) PCT No.: PCT/US2020/028387

§ 371 Date: Aug. 11, 2022

(51) Int. Cl. C12Q 1/6895

(52) U.S. Cl. CPC C12Q 1/6895; C12Q 2600/13; C12Q 2600/158

(57) ABSTRACT

The present disclosure provides a rice event GSX2-55 and a detecting and using method thereof, and provides a plant, a plant cell, a tissue or a seed thereof containing a specificity polynucleotide of the event GSX2-55. The present disclosure further provides a measuring method and kit for detecting existence of the rice event GSX2-55 based on DNA sequence of the recombinant construct inserted into rice genome and Flanking sequence of insertion site and a method for generating rice recessive cell nucleus male sterile line.

### CROSS-REFERENCES TO RELATED APPLICATION

The present application is a National Stage of International Patent Application No: PCT/US202028387 filed on Apr. 16, 2020, the entire content of which is incorporated in this application by reference.

### SEQUENCE LISTING

The present application contains a Sequence Listing which has been submitted electronically in ASCII format and is hereby incorporated by reference in its entirety. Said ASCII copy is named_Sequence_Listing.txt and is 59,084.8 bytes in size, and the Sequence listing is identical to the international application No. PCT/US202028387 filed on Apr. 16, 2020.

### TECHNICAL FIELD

The present disclosure relates to the field of the plant molecular biology and seed breeding, and in particular to a nucleic acid, a rice event GSX2-55, a method and kit for detecting the rice event GSX2-55, and a method for generating rice recessive cell nucleus male sterile line.

### BACKGROUND

Rice is the first food crop in China, and It's production status directly concerns food safety. In 2015, rice planting area in China is 456 million mu, total yield is 201 million ton, the planting area and the total yield respectively account for 27% and 36% of cereals, the rice accounts for about 60% of all food crop consumption, and rice food consumption accounts for 85% of rice total consumption. If it is calculated according to 1347 million of a population base in 2011 and 0.5% of annual average increase, it is estimated that population reaches 1408 million in 2020, and according to 140 kg of the rice by annual consumption per capita at present, the total consumption reaches 197 million ton. In addition, industrial demand to the rice is also increased in the fields of wine making, pharmacy, seasonings, feed processing and the like.

Except the yield, requirements of people to rice quality are higher and higher, especially demand growth to the high-quality rice in coastal developed areas is faster, and import demand of the high-quality rice is also increased annually. Population increase and cultivated land area decrease requires to improve rice total yield and yield per unit greatly, environment-friendliness requires to reduce utilization of water resources, pesticides and chemical fertilizers, and social civilization development and living standard improvement requires to produce the cereals with higher quality. It is a problem to be solved in China and even a global problem that how to produce more cereals with the higher quality in limited land and friendly ecological environment.

Each time of great improvement of rice production is accompanied by a breakthrough technology. In the 1960s, the ‘green revolution’ dwarfs plant height of crops of rice, wheat and corn and the like, and it is supplemented with pesticides and agricultural machinery, so the yield of the cereals is greatly increased, and food self-support problem of a developing country is solved. In the 1970s and 1980s, Chinese scientists, represented by Yuan Longping, create three-line hybrid rice system (‘three-line’ for short) by using cytoplasmic male sterile (CMS) Lines, so the rice yield is improved by 20% nearly, it is called as the second ‘green revolution’, it makes great contribution to guarantee food supply of our country, and powerful force of heterosis is also displayed to the world, crop crossbreeding has become a major route for improving the food yield.

The crossbreeding is one of the major routes for breeding new cultivars at present, and is an important method of modern breeding. Because a genetype with fine sites of parents may occur in the first generation of hybrid, In addition the epistasis interaction effect between the sites, a transgressive new individual may be screened out, and in recent decades, the heterosis has been extensively utilized as a means for improving a crop yield, improving crop quality, and improving crop anti-biont stress (resisting insects, diseases and grass and the like) and anti-abiotic stress (resisting drought, salt and a low temperature and the like). The hybridization rice breeding technology has several important links: one, a usable male sterile line; two, stably and efficiently breeding the male sterile line; three, measuring and matching the male sterile line and screening out a perfect hybridized combination; and four, efficiently producing hybrid rice seeds in low cost. One of the four links may not be omitted, and it is determined whether a commercialized hybrid rice breeding technical system may be accepted in the market and may bring social and economic benefits.

The ‘three-line’ sterile line using nucleo-cytoplasmic interaction as a core is restricted by specific restorer lines and maintainer lines, only small part of the rice germplasm resources may be used, and the screened perfect hybridized combination measured and matched by the sterile line is restricted; and the ‘two-line’ male sterile line using a photo-temperature sensitive nucleus gene as a basis is capable of overcoming the restriction of the ‘three-line’ sterile line by the specific restorer lines and maintainer lines, and improving a utilization ratio of rice resources, but fertility of the ‘two-line’ male sterile line is affected by environmental factors of light and temperature and the like, the fertility is unstable, and ‘two-line’ male sterile line reproduction and seed production high risk is existent. So, the current rice crossbreeding technical system still needs to be improved.

### SUMMARY

The present disclosure is achieved on the basis of the following discovery of the inventor: a indica rice cultivar Huanghuazhan mutant library is established by using mutagenesis of ethylmethane sulfonate (EMS), a recessive nucleus male sterile line Zhen18A is screened out and obtained, and a gene OsNP1 (Oryza sativa No Pollen1) (Chang et al., Construction of a male sterility system or hybrid rice breeding and seed production using a nuclear male sterility gene (2016) PNAS. 113(49):14145-14150) for controlling the recessive nucleus sterile character is cloned. Pollen ablation gene ZmAA (Maiza α-amylase gene; SEQ ID NO:20), Seed screening gene DsRed (Discosoma sp. red fluorescent protein; SEQ ID NO:19) and fertility restoring gene OsNP1 (SEQ ID NO:21) are introduced into the transformed receptor Zhen 18A through genetic transformation, and through analysis of multi-generation continuous molecular characteristics and Agronomic traits of thousands of positive transformants, the rice event GSX2-55 is selected preferably. The rice event GSX2-55 is used as the maintainer line, and contains a rice recessive nucleus sterile gene site (osnp1/osnp1) and an exogenous insertion sequence (DsRed/ZmAA/OsNP1), through selfing or hybridizing with the male sterile line containing the homozygous recessive nucleus sterile gene (osnp1/osnp1), rice recessive nucleus sterile line Zhen18A without containing a transgenic DNA component can be produced, and achieved the purpose to produce rice recessive nucleus male sterile lines in large scale. Further, rice recessive nucleus male sterile line Zhen18A may perform matching and production of hybrid seeds (FIG. 5) with the rice restorer line. The technology is capable of producing the non-transgenic rice recessive nucleus male sterile line by using a biotechnology method, and effectively solving the bottleneck problem faced in a rice hybrid seed production process, namely the problem that the resource utilization ratio of the three-line method is low and the sterile line fertility of the two-line method is unstable.

The expression of the exogenous gene in the plant is affected by the insertion site thereof in the plant genome, and it may be caused by regulation of a chromatin structure (such as a structure of a heterochromatin) around the insertion site or a nearby transcription regulation element (such as an enhancer) (Weising et al., Foreign genes in plants: transfer, structure, expression, and disclosures. (1988) Ann. Rev. Genet 22:421-477), for example, it may be observed that the expression levels of the exogenous genes have a greater difference between numerous events with the different insertion sites obtained by the exogenous gene transformation, and it may also be observed that the exogenous gene has a spatio-temporal expression difference existing in the different transformed strains, and this type of the different is not caused by an expression cassette constructed by an expression regulation element, such as a promoter selected artificially. At the same time, an overall phenotype of the plant may be affected by the integration of the exogenous genes in different positions of the plant genome, for example, the expression of a plant endogenous gene in the insertion site may be affected by the insertion of the exogenous gene in the plant genome. So, in a creation process of the transformation event, it is necessary to produce hundreds of the independent transformation events, through screening a large number of the transformation events, an optimal transformation event in comply with a commercialized requirement is identified and obtained, and has the exogenous gene integration site meeting the requirement and the expression level and target characters, and the adverse effect is not caused to other phenotypes and economical characters of the plant. Further, through backcross transformation method, the exogenous insertion sequence of the optimal event is transformed to other varieties of genetic background through the hybridization, and more excellent new cultivars are created.

In the plant or the seed or the offspring thereof or the offspring of sexual hybridization, the existence or inexistence of the specific detection transformation event is very important, an event specific detection method is capable of identifying a specific junction site (junction) between the inserted exogenous DNA and the transformation acceptor genome, this not only relates to the transformed exogenous DNA, but also relates to the insertion integration position thereof in the genome of the host plant or the seed. In addition, the method for detecting the specific event is very helpful to stipulate pre-marketing permission and label of transgenic plant food, monitor the environment and monitor the characters of the crops in the field and the like.

In order to achieve the above purpose, the present disclosure provides recombinant DNA molecules related to the rice event GSX2-55. These recombinant DNA molecules may contain regions of genomes DNA of representative transgenic insertion flanks, and/or regions of transgene insertion, and/or nucleotide molecules of nucleotide sequences of connection sequences (such as a connection region between the transgenic insertion fragment of the rice event GSX2-55 and the flanking genomic DNA) of any of these regions.

The term ‘event’ is a DNA molecule comprising the inserted DNA and the flanking rice genomic DNA Immediately adjacent to either side of the inserted DNA, the DNA molecule is created by the action of inserting the transgenic DNA into the genome of the rice plant, namely transformation action. So, the DNA molecule is specific to the event, and has a specific nucleotide sequence for the rice plant genome in which the transgenic DNA is inserted. Because the nucleotide sequence contains the sequences of both of a specific region of the rice genomic DNA and an insertion fragment of the transgenic DNA, the DNA inserted in the rice event GSX2-55 is specific and unique to the rice event GSX2-55 relative to arrangement modes of the genomes DNA of other rice plants. The DNA molecule is also an overall forming part of a rice chromosome containing the event GSX2-55, so the DNA molecule is static in the plant and may be transmitted to an offspring of the plant.

“Rice event GSX2-55” refers to the transformation of the tightly linked DsRed gene expression cassette, ZmAA gene expression cassette, and OsNP1 gene expression cassette into the rice genome, resulting in a 5′ junction region corresponding to the rice genome (sequence is SEQ ID NO: 1, 2, 3, 7), which is the 5′ end junction region of the rice genome and the inserted heterologous DNA molecule; the 3′ junction region corresponding to the rice genome (sequence is SEQ ID NO: 4, 5, 6, 8) DNA molecule, which is the 3′ end junction region of the rice genome and the inserted heterologous DNA molecule; the heterologous DNA molecule inserted into the genome of event GSX2-55 is represented by SEQ ID NO: 9 provided; the contiguous nucleotide sequence representing the rice event GSX2-55 containing the 5′ end junction region, the inserted heterologous DNA molecule and the 3′ end junction region are provided by SEQ ID NO: 10, and SEQ ID NO: 10 contains SEQ ID NOs: 1-9.

In the present disclosure, the rice event GSX2-55 contains an integrated transgenic expression cassette, and is endowed with a method of generating rice recessive nucleus male sterile line through selfing or hybridizing. The insertion site of the transgenic expression cassette contained in the rice event GSX2-55 is always located in a heterozygosis state in each generation, and the expression of OsNP1 gene restores fertility of rice male sterile line (osnp1/osnp1), and fertility pollen is generated. A half of the pollen contains the insertion gene (DsRed/ZmAA/OsNP1) and also contains the osnp1 gene (sterility gene site), specificity of ZmAA gene is expressed in the pollen in a mature period, energy substance starch in the pollen is degraded, and the ablation (losing fertilization capacity) of the pollen containing the exogenous gene is caused; the other half of the pollen contains the osnp1 gene, and is normal fertility. In a similar way, a half in event GSX2-55 female gamete contains the exogenous gene (DsRed/ZmAA/OsNP1) and also contains the osnp1 gene, and the other half only contains the osnp1 gene, two types of the seeds (FIG. 5) are generated by selfing, one type contains the exogenous insertion gene (DsRed/ZmAA/OsNP1;osnp1/osnp1) which is the maintainer line GSX2-55, and the other type does not contain the exogenous insertion gene which is a male sterile line (osnp1/osnp1). DsRed protein expressed in the rice event GSX2-55 seed emits red fluorescence under green light excitation, and the male sterile line seed can not emit the red fluorescence because the sterile line seed does not contain the transgenic DNA sequence and the expressed DsRed protein thereof, so the maintainer line GSX2-55 (DsRed/ZmAA/OsNP1; osnp1/osnp1) and the male sterile line (osnp1/osnp1) may be efficiently sorted through a fluoroscope or a color sorter.

While the rice recessive nucleus male sterile line (osnp1/osnp1) is used as a pollen acceptor, the rice recessive nucleus male sterile line (osnp1/osnp1) without containing the transgenic DNA is generated by hybridizing with the rice event GSX2-55.

While used in the text, the term ‘recombination’ is a form of a DNA and/or a protein and/or a living body which does not exist in the natural world generally and is generated by human intervention, such human intervention may generate a recombinant DNA molecule and/or a recombinant plant. While used in the text, the ‘recombinant DNA molecule’ is a DNA molecule of a DNA molecule combination which is contained in the natural world, does not occur together and is generated by the human intervention, for example, the DNA molecule of the combination containing at least two mutually heterogenous DNA molecules, and/or the DNA molecule containing a transgene artificially integrated to a genomic DNA of a host cell and a related flanking DNA of the host cell genome. An instance of the recombinant DNA molecule is the DNA molecule which is described in the text and obtained by enabling the transgene to be inserted into the rice genomic DNA, and it may finally cause the expression of the recombinant DNA and/or the protein molecule in the living body. While used in the text, the ‘recombinant plant’ is a plant which does not exist in the natural world and is a result of the human intervention, and contains the transgene integrated into the genome thereof and/or the heterogenous DNA molecule. As a result of such genome change, the recombinant plant is apparently different from a related wild-type plant. An instance of the recombinant plant is a rice plant which is described in the text and contains the event GSX2-55.

While used in the text, the term ‘transgene’ is a nucleotide molecule which is artificially integrated into the host cell genome. Such transgene may be heterogenous to the host cell. The term ‘transgenic plant’ is a plant containing such transgene.

While used in the text, the term ‘heterogenous’ is that a first molecule in the natural world is not combined with a second molecule generally for existence. For example, a molecule may be derived from a first species and inserted into a genome of a second species. So the molecule is heterogenous to the host, and artificially integrated into the genome of the host cell.

While used in the text, the term ‘chimeric’ is a single DNA molecule generated by fusing a first DNA molecule to a second DNA molecule, herein the first and second DNA molecules are not existent generally by the construction (namely mutual fusion). So the chimeric DNA molecule is a new DNA molecule which does not exist in the natural world generally.

While used in the text, the term ‘DNA’, ‘DNA molecule’ and ‘nucleotide molecule’ are the genome or the DNA molecule from a synthesis source, namely a polymer of a deoxyribonucleotide base or a polynucleotide molecule, and it is read from a 5′ (upstream) terminal to 3′ (downstream) terminal. While used in the text, the term ‘DNA sequence’, ‘nucleotide sequence’ or ‘polynucleotide sequence’ is a nucleotide sequence of the DNA molecule. In accordance with practice, the nucleotide sequence of the present disclosure provided as SEQ ID NO: 1-10 and the fragment thereof are only disclosed in allusion to one of two complementary nucleotide sequence chains. This hints that the complementary sequences (namely a sequence of a complementary chain, also called as a reverse complementary chain) fall within a scope of the present disclosure, and clearly fall within a scope of a subject required to be protected.

An inserted transgene DNA and the nucleotide sequence of the rice genomic DNA at any one terminal flanking of the inserted transgene DNA are provided in the text (SEQ ID NO: 10). Its sub-section is the inserted transgene DNA provided by SEQ ID NO: 9. The nucleotide sequence of the rice genomic DNA which is physically connected to the inserted transgene DNA through a phosphate diester bond and thereby adjacent to a 5′ terminal thereof is as shown in FIG. 1, and provided by SEQ ID NO: 1, 2, 3, and 7. The nucleotide sequence of the rice genomic DNA which is physically connected to the inserted transgene DNA through the phosphate diester bond and thereby adjacent to a 3′ terminal thereof is as shown in FIG. 1, and provided by SEQ ID NO: 4, 5, 6 and 8.

The rice event GSX2-55 further contains two regions, one region crosses the transgene DNA and is inserted into the 5′ position in the genome, and the other region crosses the transgene DNA and is inserted into the 3′ position in the genome, and it is respectively called as 5′ junction and 3′ junction in the text. ‘Junction sequence’ or ‘junction region’ is the DNA sequence of the cross-inserted transgene DNA and the neighboring flanking genomic DNA and/or the corresponding DNA molecule. The junction sequence may be arbitrarily shown by the nucleotide sequence provided by SEQ ID NO: 1 and SEQ ID NO: 4, and each sequence shows 10 nucleotides of the inserted transgene DNA and the neighboring flanking genomic DNA. Or, the junction sequence may be arbitrarily shown by the nucleotide sequence provided by SEQ ID NO: 2 and SEQ ID NO: 5, and each sequence shows 30 nucleotides of the inserted transgene DNA and the neighboring flanking genomic DNA. Or, the junction sequence may be arbitrarily shown by the nucleotide sequence provided by SEQ ID NO: 3 and SEQ ID NO: 6, and each sequence shows 50 nucleotides of the inserted transgene DNA and the neighboring flanking genomic DNA. These nucleotides are connected through the phosphate diester bonds, and exist in the rice event GSX2-55 as a part of the genome. In a sample derived from rice plant, seed or plant part, one or more identifications in the SEQ ID NO: 1-10 may determine that the DNA is acquired from the rice event GSX2-55, and may diagnose existence of the DNA derived from the rice event GSX2-55 in the sample. So, the present disclosure provides the DNA molecule containing at least one nucleotide sequence provided by SEQ ID NO: 1-10. Any one DNA fragment derived from the transgenic rice event GSX2-55 which contains at least one sequence provided by the SEQ ID NO: 1-10 falls within a scope of the present disclosure. In addition, any polynucleotides of the complementary sequences of any sequences described in the paragraph fall within the scope of the present disclosure. FIG. 1 shows arrangement of SEQ ID NO: 1-9 from 5′ to 3′ relative to the physical arrangement of the SEQ IN NO: 10.

The present disclosure provides a DNA molecule which may be used as a primer and a probe for diagnosing the rice event GSX2-55 and an amplicon for diagnosing existence of the rice event GSX2-55. The present disclosure further discloses rice plant, plant cell, plant part, commodity, offspring and seed containing these molecules.

The above primer or probe is specific to the target nucleotide sequence, and thereby may be used for identifying the nucleotide sequence of the rice event GSX2-55 through the method of the present disclosure described in the text.

The ‘primer’ is the highly-purified separated polynucleotide generally, and it is designed for a specificity annealing or hybridizing method related to thermal amplification. A pair of the primers may be used in the thermal amplification, for example polymerase chain reaction (PCR), with template DNA together, for example rice genomic DNA sample, as to generate an amplicon. While used in the text, the ‘amplicon’ is DNA chip or fragment synthesized by using an amplification technology. The amplicon of the present disclosure contains at least one sequence provided by SEQ ID NO: 1-10. The primer is generally designed to be a hybrid which is hybridized with a complementary target DNA chain so as to form between the primer and the target DNA chain, and existence of the primer is an identification site that polymerase uses the target DNA chain as a template for starting primer extension. An exemplary DNA which may be used as the primer is provided by SEQ ID NO: 11, SEQ ID NO: 13, SEQ ID NO: 15 and SEQ ID NO: 17, primer pairs provided by the SEQ ID NO: 11 and the SEQ ID NO: 13 may be used as a first DNA molecule and a second DNA molecule different from the first DNA molecule, and each of SEQ ID NO: 11 and SEQ ID NO: 13 has an enough long continuous nucleotide of the SEQ ID NO: 10 so as to achieve the effect that the DNA primer for diagnosing the amplicon of the sample rice event GSX2-55 DNA is generated while used with the template DNA derived from the rice event GSX2-55 together in a thermal amplification reaction.

The ‘probe’ is a separated nucleic acid complementary with the target nucleotide chain. The probe of the present disclosure includes a desoxyribonucleic acid or a ribonucleic acid, and further includes other probe materials specifically bonded to a target DNA sequence, and detection of this bonding may be used for diagnosing, identifying, determining or verifying existence of the target DNA sequence in a specific sample. The probe may be connected to a conventional detectable label or a report molecule, for example radioactive isotope, ligand, chemical luminescence agent or enzyme. An exemplary DNA molecule which may be used as the probe is provided by SEQ ID NO: 12 and SEQ ID NO: 16.

The probe and the primer in accordance with the present disclosure may have complete sequence homoousia with the target sequence, although the primer and the probe which keep preference hybridization capacity with the target sequence and are different from the target sequence may be designed through a conventional method. In order to enable a nucleic acid molecule to achieve the effect of the primer and the probe, enough complementation only needs to be performed in the sequence so that a stable double-chain structure is formed in a used specific solvent and salt concentration. Existence of the transgenic DNA derived from the rice event GSX2-55 in the sample may be identified by using any conventional nucleic acid hybridization or amplification methods. A length of the probe and the primer is at least 11 nucleotides generally, at least about 18 nucleotides, at least about 24 nucleotides or at least about 30 nucleotides or longer. Such probe and primer are specifically hybridized with the target DNA sequence in a strict hybridization condition. The conventional stringent condition is described in Sambrook and the like, 1989 and Haymes and the like, Nucleic Acid Hybridization, APractical Approach, IRL Press, Washington, D.C. (1985). While used in the text, if two nucleic acid molecules may form an anti-parallel double-chain nucleic acid structure, the two molecules may be mutually specific-hybridized. If two nucleic acid molecules show complete complementarity, one nucleic acid molecule is a ‘complementary’ of the other nucleic acid molecule. While used in the text, if while two molecules are compared, each nucleotide of the first molecule is complementary with each nucleotide of the second molecule, the two molecules show ‘complete complementarity’. If two molecules may be mutually hybridized by enough stability so as to allow them to keep mutual annealing in at least conventional ‘low stringency’ condition, the two molecules are ‘lowest complementary’. Similarly, if the molecules may be mutually hybridized by the enough stability so as to allow them to keep the mutual annealing in a conventional ‘high stringency’ condition, they are ‘complementary’. So, deviation of the complete complementarity is allowed, only if such deviation does not completely exclude capacity of the molecule for forming the double-chain structure.

A large number of methods known by those skilled in the art may be used for separating and operating the DNA molecule or the fragment thereof disclosed in the present disclosure. For example, polymerase chain reaction (PCR) technology may be used for amplifying a specific starting DNA molecule and/or generating a mutant of the original molecule. The DNA molecule or the fragment thereof may also be obtained through other technologies, for example the fragment is directly synthesized through chemical means, and it is exactly realized by using an automatic oligonucleotide synthesizer generally.

So, except other purposes, the DNA molecule and the corresponding nucleotide sequence provided in the text may be used for identifying the rice event GSX2-55, selecting a plant cultivar or hybrid containing the rice event GSX2-55, detecting existence of the DNA derived from the rice event GSX2-55 in a sample and monitoring the rice event GSX2-55 in the sample or existence and/or inexistence of the plant part derived from rice plant containing the event GSX2-55.

The rice plant, the plant cell, the plant part (for example pollen, fruit, spike tissue, flower tissue, root tissue, stem tissue and leaf tissue), the commodity, the offspring and the seed provided by the present disclosure contain a detectable amount of the polynucleotide of the present disclosure, for example, the polynucleotide of at least one sequence is provided by the SEQ ID NO: 1-10. The plant, the offspring, the seed, the plant cell and the plant part of the present disclosure may also contain one or more other transgenes. Such transgene may be any nucleotide sequence encoding the protein or RNA molecule with required characters, the characters include, but not limited to, improved insect resistance, improved water utilization efficiency, improved yield performance, improved drought resistance, improved seed quality, nutritional quality of the characters and/or improved herbicide tolerance, herein the required characters are measured relative to the rice plant lack of such other transgenes.

The plant of the present disclosure may transmit the event DNA (including the transgene) to the offspring. While used in the text, the ‘offspring’ includes the event DNA derived from an ancestor plant and/or any plant, seed, plant cell and/or regenerated plant part containing the DNA molecule with at least one sequence selected from the SEQ ID NO: 1-10. The transgene may be homozygous or heterozygous in the plant, the offspring and the seed. The offspring may be grown from the seed generated by the plant containing the rice event GSX2-55 and/or from the seed generated by the plant pollinated by the pollen of the plant containing the rice event GSX2-55.

An offspring plant may be self-pollination or cross-pollination, for example, mutant or hybrid seed or plant is generated by hybridizing with the other type of the plant without a genetic relationship. The other type of the plant without the genetic relationship may be transgenic or non-transgenic. So, the mutant or hybrid seed or plant of the present disclosure may be generated by enabling a first parent of the specific and unique DNA lack of the rice event GSX2-55 to be hybridized with a second parent containing the rice event GSX2-55, so that a hybrid of the specific and unique DNA containing the rice event GSX2-55 is generated. Each parent may be the hybrid or inbred line/mutant, only if the plant or seed of the present disclosure is generated by hybridizing or breeding, namely it has at least one allele of the DNA containing the rice event GSX2-55 and/or has the seed of the DNA molecule with at least one sequence selected from the SEQ IN NO: 1-10. So, two different transgenic plants may be hybridized to generate the hybrid offspring containing two independently separated added exogenous genes.

While used in the text, the plant part, provided by the present disclosure, derived from the rice plant containing the event GSX2-55 is any part of the plant formed by a material derived from the rice plant containing the event GSX2-55. The plant part includes, but not limited to, the pollen, the fruit, the spike tissue, the flower tissue, the root tissue, the stem tissue and the leaf tissue. The plant part may be survivable, non-survivable, regenerated and/or non-regenerated.

While used in the text, the ‘commodity’ provided by the present disclosure is any combination or product formed by a material derived from the plant, the seed, the plant cell or the plant part containing the rice event GSX2-55. The commodity may be sold to a consumer, and may be survivable or non-survivable. The non-survivable commodity includes, but not limited to, the non-survivable seed, the processed seed, the seed part and the plant part, and is used for a feed, food, and oil. The survivable commodity includes, but not limited to, the seed, the plant and the plant cell. So, the rice plant containing the event GSX2-55 may be used for manufacturing any commodity obtained from the rice generally. Any such commodity derived from the rice plant containing the event GSX2-55 may contain at least detectable specific and unique DNA corresponding to the rice event GSX2-55, and particularly may contain detectable the polynucleotide containing the DNA molecule with at least one sequence selected from the SEQ ID NO: 1-10. Any standard method for detecting the nucleotide molecule may be used, including a detection method disclosed in the text. If any detectable DNA molecule with at least one sequence selected from the SEQ ID NO: 1-10 exists in the commodity, the commodity falls within the scope of the present disclosure.

So, except other purposes, the plant, the offspring, the seed, the plant cell, the plant part and the commodity of the present disclosure may be used for growing the plant, and a purpose thereof is to generate the seed and/or the plant part containing the rice event GSX2-55 so as to achieve an agricultural purpose, the offspring containing the rice event GSX2-55 is generated and used for plant breeding and researching purpose, used for industrial and research disclosures by biology technology, and used for commodity is sold to the consumer.

The present disclosure provides a method for detecting existence and/or inexistence of the DNA derived from the rice event GSX2-55, and thereby provides a method, a combination and a kit for detecting existence and/or inexistence of the rice event GSX2-55. The present disclosure provides a method for, in a nucleic acid amplification reaction of the genomic DNA containing the rice event GSX2-55 plant or seed, contacting with a primer group for generating an amplicon for diagnosing the rice event GSX2-55, and performing the nucleic acid amplification reaction and thereby generating the amplicon, and detecting existence and/or inexistence of the amplicon so as to detect the rice event GSX2-55.

The above method is formed by the following steps: (I) a DNA sample is extracted from at least one rice cell, tissue, seed and plant, (II) the DNA sample contacts with a DNA probe which is specific to the event GSX2-55, (Ill) the probe is allowed to be hybridized with the DNA sample in a strict hybridization condition, and (IV) the hybridization between the probe and the target DNA sample is detected.

The above method may be also formed by the following steps: (I) the DNA sample is extracted from at least one rice cell, tissue, seed or plant, (II) the DNA sample contacts with a primer pair of the amplicon for generating the event GSX2-55 in a condition suitable for DNA amplification, (Ill) a DNA amplification reaction is performed, and (IV) the amplicon molecule is detected and/or it is verified that the nucleotide sequence of the amplicon is the nucleotide sequence containing the specificity of the event GSX2-55, for example one nucleotide sequence selected from the SEQ IN NO: 1-10. The amplicon should be the amplicon specific to the event GSX2-55, for example one nucleotide sequence selected from the SEQ IN NO: 1-10. Detection of the nucleotide sequence specific to the event GSX2-55 in the amplicon is deterministic and/or diagnostic to existence of the specific DNA of the rice event GSX2-55 in the sample. An instance of the primer pair of the amplicon for generating the event GSX2-55 DNA in the condition suitable for the DNA amplification is provided by the SEQ ID NO: 11 and the SEQ ID NO: 13. Other primer pairs may be easily designed by those skilled in the art, and should contain at least one fragment of the SEQ IN NO: 10.

The DNA detection kit provided by the above two methods may be used for identifying the rice event GSX2-55 DNA in the sample. Such kit contains a DNA primer and/or probe containing the fragment of the SEQ ID NO: 1-10. An instance of such kit contains at least one DNA molecule with an enough long continuous nucleotide of the SEQ ID NO: 10, as to achieve the effect of the DNA probe for detecting existence and/or inexistence of the DNA derived from the transgenic rice plant containing the event GSX2-55. The DNA derived from the transgenic rice plant containing the event GSX2-55 contains the DNA molecule with at least one sequence selected from the SEQ ID NO: 1-10. The DNA molecule which is used as the DNA probe enough is provided, and it may be used for detecting or diagnosing existence and/or inexistence of the rice event GSX2-55 in the sample. Other probes may be easily designed by those skilled in the art, and should contain at least 15 continuous nucleotides of the SEQ ID NO: 10 and are unique enough to the rice event GSX2-55, as to identify the DNA derived from the event. The other type of the kit contains the primer pair for generating the amplicon, the amplicon may be used for detecting existence and/or inexistence of the DNA derived from the rice event GSX2-55 in the sample. Such kit utilizes the method including the following steps: the target DNA sample contacts with the primer pair described in the text, after that, the nucleic acid amplification reaction which is enough to generate the amplicon of the DNA molecule with at least one sequence selected from the SEQ IN NO: 1-10 is performed, and existence and/or inexistence of the amplicon is detected. Such method may further includes a step of sequencing the amplicon or the fragment thereof, the existence thereof to the specific DNA of the rice event GSX2-55 in the target DNA sample is deterministic, namely the existence of the specific DNA of the rice event GSX2-55 in the target DNA sample is diagnosed. Other primer pairs may be easily designed by those skilled in the art, and should contain at least 15 continuous nucleotides of the SEQ ID NO: 10 and are unique enough to the rice event GSX2-55 DNA so as to identify the DNA derived from the event.

Nucleic acid amplification may be performed through each known nucleic acid amplification method in the field, including the thermal amplification method. A large number of technologies for detecting, quantifying and/or sequencing the amplicon generated through these methods are known in the field. An exemplary technology for implementing the present disclosure is TAQMAN® (PE Applies Biosystems, Foster City, Calif.).

Except other aspects, the kit and the detection method of the present disclosure may be used for identifying the rice event GSX2-55, selecting the plant cultivar containing the rice event GSX2-55, detecting existence of the DNA derived from the transgenic rice plant containing the event GSX2-55 in the sample, and detecting the rice plant containing the event GSX2-55 in the sample or existence and/or inexistence of the plant part derived from the rice plant containing the event GSX2-55.

The sequence, the junction sequence or the flanking sequence of the heterogenous DNA insertion fragment from the rice event GSX2-55 may be verified by amplifying such sequence from the event through using the primer derived from the sequence provided by the text, and performing the standard DNA sequencing on the amplicon or the cloned DNA.

While used in the text, the term ‘containing’ means ‘including but not limited to’.

The present disclosure provides the above rice plant, seed, plant cell, offspring plant, plant part or commodity derived from the plant, the plant cell or the seed containing the rice event GSX2-55 inserted in a specific position of the plant genome, such offspring may be generated by sexual hybridization between the rice event GSX2-55 or the offspring thereof and the other plant. Such other plant may be a transgenic plant and/or non-transgenic plant containing same or different transgenes. Even if repeatedly backcrossed with recurrent parent, the insertion DNA and the flanking DNA from the transformed parent also exist in a same genome position in the hybrid offspring. Specifically, the present disclosure provides the rice plant, the seed, the plant cell, the offspring plant, the plant part or the commodity containing the recombinant DNA molecule, the recombinant DNA molecule has the nucleotide sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9 and SEQ ID NO:10 and complementary sequences and fragments thereof. The present disclosure further provides the rice plant, the seed, the plant cell, the offspring plant, the plant part or the commodity derived from the plant or the seed containing the rice event GSX2-55 and containing the recombinant DNA molecule, the recombinant DNA molecule generates the DNA molecule of the amplification containing the sequence selected from the SEQ ID NO: 1-10 in the DNA amplification method.

The present disclosure further provides a method for generating the rice seed through the rice event GSX2-55, and more specifically provides a method for generating rice recessive nucleus male sterile line. The event contains single insertion of the transgenic DNA in chromosome/genome of rice germplasm. The ‘event’ is generated by the following steps: (I) the plant cell is transformed by using a nucleic acid construct including a target transgene; (II) a plant population which is acquired by the insertion of the transgene in the plant genome is regenerated; and (III) a specific position in which the transgene is inserted into the plant genome is selected as a specific plant of the characters. Specifically, the method includes the following steps: the step of planting the rice plant containing the event GSX2-55 in the field, herein the rice plant containing the event GSX2-55 contains a mutant osnp1/osnp1 gene without a fertility function, and through selfing of the rice plant containing the event GSX2-55, or using the rice event GSX2-55 as a male parent for performing the sexual hybridization with a second rice plant, the second rice plant contains the mutant osnp1/osnp1 gene without the fertility function; the step of selfing or hybridizing the seed of the rice harvested from the field; and the step of selecting the offspring seed of non-red fluorescence through a fluorescence sorter and/or manual work. Because the seed containing the transgenic rice event GSX2-55 emits the red fluorescence under green light, the non-red fluorescence seed is the recessive nucleus male sterile line of the rice, and does not emit the red fluorescence since the osnp1/osnp1 gene mutation site is contained.

While used in the text, the term ‘rice’ is Oryza sativa L. ssp. Indica, and contains all plant varieties which may perform breeding with the rice, and contains a wild rice species and a plant which allows interspecific breeding and belongs to Oryza L.

The commodity of the plant, the offspring, the seed, the plant cell and the plant part of the present disclosure may evaluate DNA formation, gene expression and/or protein expression thereof. Such evaluation may be performed through using any standard method, for example PCR, Northern blot, Southern blot, Western blot, immunoprecipitation and ELISA, or using the detection method and/or the detection kit provided in the text.

The present disclosure provides a method for diagnosing junction of the plant or seed containing the rice event GSX2-55, the method includes the following steps: the rice DNA sample contacts with the primer group containing SEQ ID NO:11, SEQ ID NO:13, SEQ ID NO:15 and SEQ ID NO:17 and the probe group containing SEQ ID NO 12 and SEQ ID NO:16; after that, nucleic acid amplification reaction of the sample, the primer group and the probe group is performed; after that, in the nucleic acid amplification reaction, a first fluorescence signal for diagnosing the event GSX2-55 and a second fluorescence signal which is different from the first fluorescence signal and is used for diagnosing the rice genomic DNA are detected; and existence and/or inexistence of the first fluorescence signal and the second fluorescence signal in the nucleic acid amplification reaction is analyzed, herein if two fluorescence signals are existent, it is indicated that the detected sample contains the event GSX2-55.

The beneficial effect which is achieved by the present disclosure is as follows:

1. a method capable of effectively and stably breeding rice recessive nucleus male sterile line is provided; and

2. a method for rice crossbreeding by adequately utilizing rice germplasm resources and improving hybrid rice breeding efficiency thereof is provided.

### BRIEF DESCRIPTION OF THE DRAWINGS

FIG. 1 is a formation of transgenic insertion fragment in a genome of a rice plant containing an event GSX2-55, herein [A] corresponds to a relative position of SEQ ID NO: 1; [B] corresponds to a relative position of SEQ ID NO: 2; [C] corresponds to a relative position of SEQ ID NO: 3; [D] corresponds to a relative position of SEQ ID NO: 4; [E] corresponds to a relative position of SEQ ID NO: 5; [F] corresponds to a relative position of SEQ ID NO: 6; [G] corresponds to a relative position of SEQ ID NO: 7; [H] corresponds to a relative position of SEQ ID NO: 8; [I] corresponds to a relative position of SEQ ID NO: 9; [J] corresponds to a relative position of SEQ ID NO: 10; [K] corresponds to a relative position of SEQ ID NO: 11; [L] corresponds to a relative position of SEQ ID NO: 12; [M] corresponds to a relative position of SEQ ID NO: 13; [N] corresponds to a relative position of SEQ ID NO: 14; [O] corresponds to a relative position (DsRed gene expression cassette) of SEQ ID NO: 19; [P] corresponds to a relative position (ZmAA gene expression cassette) of SEQ ID NO: 20; and [Q] corresponds to a relative position (OsNP1 gene expression cassette) of SEQ ID NO: 21.

FIG. 2 is sensitivity of detecting the rice event GSX2-55 by quantitative PCR.

FIG. 3 is contrast of the rice event GSX2-55 seed in a white light state and an excited state respectively, herein the rice event GSX2-55 seed shows the red fluorescence in the excited state.

FIG. 4 is the rice event GSX2-55 (left) and contrast Huanghuazhan (right) pollen potassium iodide dyeing.

FIG. 5 is a schematic diagram of producing a rice recessive nucleus male sterile line and hybrid rice by the rice event GSX2-55.

### BRIEF DESCRIPTION OF SEQUENCES

SEQ ID NO: 1 is a sequence of 20 nucleotides, and it shows a5′ connection region of the rice genomic DNA and the integrated transgenic expression cassette. The SEQ ID NO: 1 is positioned in 957-976 bp of a nucleotide position in SEQ ID NO: 10.

SEQ ID NO: 2 is a sequence of 60 nucleotides, and it shows a 5′ connection region of the rice genomic DNA and the integrated transgenic expression cassette. The SEQ ID NO: 2 is positioned in 937-996 bp of a nucleotide position in the SEQ ID NO: 10.

SEQ ID NO: 3 is a sequence of 100 nucleotides, and it shows a 5′ connection region of the rice genomic DNA and the integrated transgenic expression cassette. The SEQ ID NO: 3 is positioned in 917-1016 bp of a nucleotide position in the SEQ ID NO: 10.

SEQ ID NO: 4 is a sequence of 20 nucleotides, and it shows a 3′ connection region of the rice genomic DNA and the integrated transgenic expression cassette. The SEQ ID NO: 4 is positioned in 13555-13574 bp of a nucleotide position in the SEQ ID NO: 10.

SEQ ID NO: 5 is a sequence of 60 nucleotides, and it shows a 3′ connection region of the rice genomic DNA and the integrated transgenic expression cassette. The SEQ ID NO: 5 is positioned in 13535-13594 bp of a nucleotide position in the SEQ ID NO: 10.

SEQ ID NO: 6 is a sequence of 100 nucleotides, and it shows a 3′ connection region of the rice genomic DNA and the integrated transgenic expression cassette. The SEQ ID NO: 6 is positioned in 13515-13614 bp of a nucleotide position in the SEQ ID NO: 10.

SEQ ID NO: 7 is a sequence of 1386 nucleotides, and it shows a 5′ connection region of the rice genomic DNA and the integrated transgenic expression cassette. The SEQ ID NO: 7 is positioned in 1-1386 bp of a nucleotide position in the SEQ ID NO: 10.

SEQ ID NO: 8 is a sequence of 1440 nucleotides, and it shows a 3′ connection region of the rice genomic DNA and the integrated transgenic expression cassette. The SEQ ID NO: 8 is positioned in 13002-14441 bp of a nucleotide position in the SEQ ID NO: 10.

SEQ ID NO: 9 is a sequence of 12598 nucleotides, and it shows an integrated transgenic expression cassette sequence. The SEQ ID NO: 9 is positioned in 967-13564 bp of a nucleotide position in the SEQ ID NO: 10.

SEQ ID NO: 10 is a continuous nucleotide sequence of a 5′ sequence of a side-adjacent inserted DNA, the integrated transgenic expression cassette sequence and a 3′ sequence of the side-adjacent inserted DNA, and the SEQ ID NO: 10 contains the SEQ ID NO: 1-9.

SEQ ID NO: 11 is a nucleotide sequence of primer TTY-55F of the rice event GSX2-55 identified by using TAQMAN® method, and is complementary with an expression cassette sequence inserted at the 3′ terminal. A primer combination TTY-55F and TTY-55R (SEQ ID NO: 13) is amplified in TAQMAN® (Applied Biosystems) so as to generate a PCR amplicon and diagnose the rice event GSX2-55.

SEQ ID NO: 12 is a nucleotide sequence of probe TP55 of the rice event GSX2-55 identified by using TAQMAN® method, and it is complementary with a sequence of a 3′ terminal junction region, the 5′ terminal of probe TP55 performs 6-FAM™ labelling, and 3′ terminal performs Minor Groove Binder (MGB) labelling, a primer combination TTY-55F and TTY-55R (SEQ ID NO: 11 and SEQ ID NO: 13), in an amplification reaction combined with the TP55 probe, releases a fluorescence signal and diagnoses the rice event GSX2-55.

SEQ ID NO: 13 is a nucleotide sequence of primer TTY-55R of the rice event GSX2-55 identified by using TAQMAN® method, and is complementary with a rice genome sequence of the 3′ terminal. A primer combination TTY-55F (SEQ ID NO: 11) and TTY-55R is amplified in the TAQMAN® (Applied Biosystems) so as to generate the PCR amplicon and diagnose the rice event GSX2-55.

SEQ ID NO: 14 is a nucleotide sequence of the PCR amplicon generated by amplifying the combination of the primers TTY-55F and TTY-55R from the TAQMAN® (Applied Biosystems).

SEQ ID NO: 15 is a nucleotide sequence of primer TSPS-F of the rice endogenous SPS (Sucrose Phosphate Synthase) gene identified by using the TAQMAN® method. A combination of the primer combination TSPS-F and TSPS-R (SEQ ID NO: 17) is amplified from the TAQMAN® (Applied Biosystems) so as to generate the PCR amplicon, and identity whether the rice genome sequence is existent.

SEQ ID NO: 16 is a nucleotide sequence of probe TPSPS of the rice endogenous SPS gene identified by using the TAQMAN® method, the 5′ terminal of the probe TPSPS performs VIC™ labelling, and 3′ terminal performs Minor Groove Binder (MGB) labeling, in the amplification reaction of using TSPS-F and TSPS-R (SEQ ID NO: 15 and SEQ ID NO: 17) to be combined with the probe TP55, the fluorescence signal is released and it is diagnosed whether the rice genome sequence is existent.

SEQ ID NO: 17 is a nucleotide sequence of primer TSPS-R of the rice endogenous SPS gene identified by using the TAQMAN® method. A combination of the primers TSPS-F and TSPS-R is amplified from the TAQMAN® (Applied Biosystems) so as to generate the PCR amplicon, and identity whether the rice genome sequence is existent.

SEQ ID NO: 18 is a nucleotide sequence of the PCR amplicon generated by amplifying the combination of the primers TSPS-F and TSPS-R (SEQ ID NO: 15 and SEQ ID NO: 17) from the TAQMAN® (Applied Biosystems).

SEQ ID NO: 19 is the DsRed gene expression cassette nucleotide sequence.

SEQ ID NO: 20 is the ZmAA gene expression cassette nucleotide sequence.

SEQ ID NO: 21 is the OsNP1 gene expression cassette nucleotide sequence.

### DETAILED DESCRIPTION OF THE EMBODIMENTS

Unless otherwise specified, methods used in the following embodiments are common methods recorded in molecular biology, tissue culture technology and agricultural handbook. For example, the specific steps may see: Molecular Cloning: A Laboratory Manual (3rd edition) (Sambrook, J., Russell, David W., 2001, Cold Spring Harbor), Plant Propagation by Tissue Culture (Edwin F. George, Michael A. Hall, Geert-Jan DeKlerk, 2008, Springer).

The following embodiments are contained to illuminate instances of some preferable implementation modes of the present disclosure. Those skilled in the art should recognize that technologies disclosed in the following embodiments represent a mode that the inventor discovers the instances which is expressed well in the embodiments of the present disclosure and thereby regarded as the preferable implementation modes thereof. However, according to the present disclosure, those skilled in the art should recognize that many variations may be made in the disclosed specific implementation mode and analogous or similar results are still acquired without departing from spirit and scope of the present disclosure.

### Embodiment 1: Rice Transformation and Selection of the Rice Event GSX2-55

The embodiment describes generation, analysis and selection of the rice event GSX2-55. Specifically, agrobacterium-mediated method is used for performing co-transformation on rice (Hiei et al., Efficient transformation of rice mediated by Agrobacterium and sequence analysis of the boundaries of the T-DNA. (1994) Plant J6(2):271-282, it is incorporated into the text by reference), Zhen18A is used as a transformation acceptor material, and agrobacterium is used for importing DsRed (SEQ ID NO: 19), ZmAA (SEQ ID NO: 20) and OsNP1 gene (SEQ ID NO: 21) nucleotide sequences into the Zhen18A (Chang et al., Construction of a male sterility system for hybrid rice breeding and seed production using a nuclear male sterility gene (2016) PNAS. 113(49):14145-14150.Fig.S7), as to obtain 1000 strains positive transgenic material or more. After that, through copy number analysis, vector backbone analysis and insertion site analysis, and agronomic traits (including seed setting rate, seed fluorescence segregation ratio, pollen inactivation rate, plant height, growing period and the like), the rice event GSX2-55 is preferably selected therefrom.

### Embodiment 2: Pollen Fertility Detection of the Rice Event GSX2-55

Pollen activity observation analysis is performed on the rice event GSX2-55 and non-transgenic controlled rice (Hemeizhan and Huanghuazhan) in the embodiment 1. Specifically, in a later blooming period of the rice, a single plant is randomly extracted from the above three varieties, one adequate mature flower bud to be bloomed is taken from each plant, and an anther is taken from each flower, the anther is placed on a glass slide, and a drop of distilled water is added, after pollen grains are released by tweezers, 1-2 drops of 1₂-KI (enabling 2 g of KI and 1 g of 1₂ to be dissolved in 300 ml of the distilled water) solution is added, covered by cover glass, and observed under a low-magnification microscope, the number of dyeing pollens and the number of non-dyeing pollens are counted. If the pollen grains are blue, it is indicated that the pollen grains contain starch and activity is stronger; and if the pollen grains are snuff colored, it is indicated that the pollen grains (FIG. 4) are hypogenetic. The experiment counts and analyses materials for 10 times repeatedly, namely 10 glass slides, one view is taken from each sheet, a standard deviation (See Table 1) of 10 times of a repeated average number is counted, and a diameter of the dyeing pollen is measured.

It is indicated from an experimental result that a probability of the rice event GSX2-55 dyed by the I₂-KI is 51.68±2.89%, and far lower than the Hemeizhan (98.02±1.19%) and Huanghuazhan (98.28±1.47%). Theoretically, a transgenic element of the rice event GSX2-55 is positioned in a heterozygosis state in each generation, so a half of the pollen does not contain an exogenous gene (fertility), and a half of the pollen contains the transgenic element (sterility), ZmAA gene contained in the element is specific-expressed in the pollen, so starch in the pollen is effectively degraded, the pollen containing the transgenic element may not be dyed by the I₂-KI, and pollen activity and fertility capacity are remarkably reduced, so transgenic pollen inactivation is caused.

TABLE 1
Pollen I₂-KI dyeing experimental data statistics

| | GSX2-55 | | | Hemeizhan | | |
|---|---|---|---|---|---|---|
| | Dyeing | Non-dyeing | | Dyeing | Non-dyeing | |
| | pollen | pollen | Dyeing | pollen | pollen | Dyeing |
| Plant number | number | number | rate | number | number | rate |
| Repetition 1 | 66 | 58 | 53.23% | 137 | 4 | 97.16% |
| Repetition 2 | 76 | 70 | 52.05% | 79 | 2 | 97.53% |
| Repetition 3 | 92 | 66 | 58.23% | 166 | 1 | 99.40% |
| Repetition 4 | 76 | 73 | 51.01% | 132 | 1 | 99.25% |
| Repetition 5 | 98 | 101 | 49.25% | 101 | 2 | 98.06% |
| Repetition 6 | 95 | 101 | 48.47% | 116 | 4 | 96.67% |
| Repetition 7 | 68 | 73 | 48.23% | 152 | 2 | 98.70% |
| Repetition 8 | 131 | 114 | 53.47% | 94 | 3 | 96.91% |
| Repetition 9 | 76 | 77 | 49.67% | 137 | 0 | 100.00% |
| Repetition 10 | 92 | 81 | 53.18% | 138 | 5 | 96.50% |

| | | |
|---|---|---|
| Ryeing rate | 51.68 ± 2.89% | 98.02 ± 1.19% |
| average value | | |

| | Huanghuazhan | | |
|---|---|---|---|
| | Dyeing | Non-dyeing | |
| | pollen | pollen | Dyeing |
| Plant number | number | number | rate |
| Repetition 1 | 114 | 0 | 100.00% |
| Repetition 2 | 138 | 0 | 100.00% |
| Repetition 3 | 135 | 1 | 99.26% |
| Repetition 4 | 162 | 4 | 97.59% |
| Repetition 5 | 180 | 3 | 98.36% |
| Repetition 6 | 149 | 4 | 97.39% |
| Repetition 7 | 153 | 0 | 100.00% |
| Repetition 8 | 102 | 5 | 95.33% |
| Repetition 9 | 130 | 3 | 97.74% |
| Repetition 10 | 133 | 4 | 97.08% |

| | |
|---|---|
| Ryeing rate | 98.28 ± 1.47% |
| average value | |

### Embodiment 3: Statistic Analysis of Fluorescence Seed and Non-Fluorescence Seed Separation Proportion

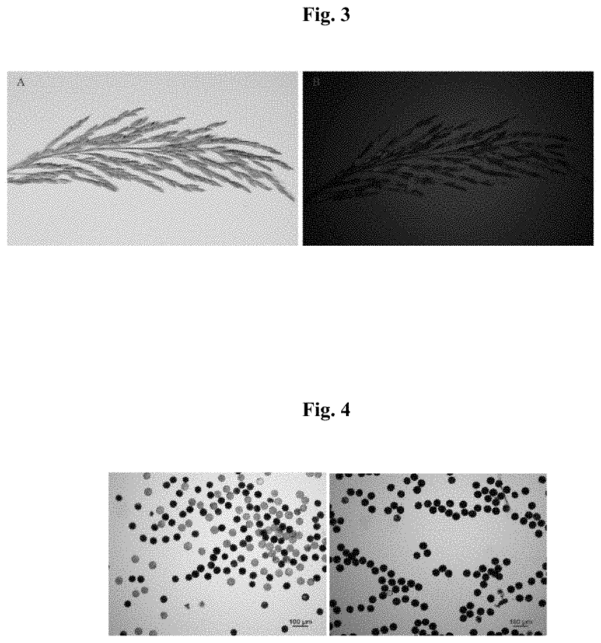

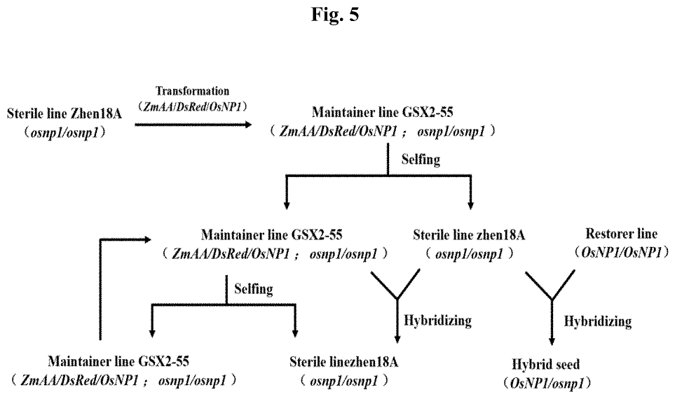

DsRed gene may efficiently express red fluorescence protein in the seed of event GSX2-55, under excitation of green light, red fluorescence emitted by the DsRed protein may be used for distinguishing the rice event GSX2-55 and the rice recessive nucleus male sterile line Zhen18A seed (FIG. 3). The rice event GSX2-55 is selfing-fruited, after threshing, impurity removal and color sorting, red fluorescence seeds (GSX2-55) and non-fluorescence seeds (Zhen18A) in GSX2-55 selfing-fruited seeds are separated, and a grain number thereof is counted.

T₄, T₅ and T₆-generation single plants (30 single plants are counted in each generation) of the rice event GSX2-55 obtained in the embodiment 1 are randomly selected, a separation rate of the harvested fluorescence seeds and non-fluorescence seeds thereof is calculated. A result is as shown in Table 2, a Chi-square X² value (df=1) of the fluorescence separation proportion of all single plants is lower than a critical value 3.81, it is indicated that the separation rates of the fluorescence seeds and non-fluorescence seeds in T₄, T₅ and T₆ three generations of the rice event GSX2-55 are in comply with 1:1.

TABLE 2
T₄-T₆ generation fluorescence separation rate data statistic
analysis (Chi-square test) of the rice event GSX2-55

| | T4 | | | T5 | | |
|---|---|---|---|---|---|---|
| | | non- | | | non- | |
| | Fluorescence | fluorescence | | fluorescence | fluorescence | |
| Plant number | seed | seed | X^{2★} | seed | seed | X^{2★} |
| Repetition 1 | 626 | 596 | 0.688 | 505 | 508 | 0.004 |
| | 752 | 756 | 0.006 | 464 | 471 | 0.039 |
| | 284 | 298 | 0.290 | 455 | 492 | 1.369 |
| | 295 | 341 | 3.184 | 511 | 483 | 0.733 |
| | 487 | 510 | 0.485 | 411 | 424 | 0.172 |
| | 715 | 732 | 0.177 | 563 | 553 | 0.073 |
| | 263 | 283 | 0.661 | 337 | 333 | 0.013 |
| | 300 | 333 | 1.618 | 251 | 283 | 1.800 |
| | 376 | 389 | 0.188 | 306 | 334 | 1.139 |
| | 550 | 527 | 0.449 | 461 | 495 | 1.139 |
| Repetition 2 | 530 | 576 | 1.831 | 265 | 306 | 2.802 |
| | 518 | 485 | 1.021 | 397 | 419 | 0.540 |
| | 444 | 469 | 0.631 | 423 | 436 | 0.168 |
| | 185 | 185 | 0.003 | 341 | 354 | 0.207 |
| | 310 | 355 | 2.911 | 330 | 347 | 0.378 |
| | 365 | 399 | 1.425 | 511 | 482 | 0.790 |
| | 285 | 276 | 0.114 | 432 | 468 | 1.361 |
| | 416 | 390 | 0.775 | 383 | 373 | 0.107 |
| | 442 | 484 | 1.815 | 431 | 426 | 0.019 |
| | 365 | 373 | 0.066 | 532 | 510 | 0.423 |
| Repetition 3 | 472 | 516 | 1.871 | 566 | 536 | 0.763 |
| | 693 | 651 | 1.251 | 415 | 436 | 0.470 |
| | 534 | 509 | 0.552 | 451 | 440 | 0.112 |
| | 409 | 414 | 0.019 | 414 | 434 | 0.426 |
| | 638 | 593 | 1.573 | 421 | 386 | 1.432 |
| | 403 | 423 | 0.437 | 325 | 366 | 2.315 |
| | 356 | 370 | 0.233 | 384 | 365 | 0.433 |
| | 488 | 493 | 0.016 | 498 | 537 | 1.395 |
| | 392 | 436 | 2.233 | 446 | 481 | 1.247 |
| | 330 | 338 | 0.073 | 406 | 415 | 0.078 |

| | T6 | | |
|---|---|---|---|
| | | non- | |
| | Fluorescence | fluorescence | |
| Plant number | seed | seed | X^{2★} |
| Repetition 1 | 1153 | 1172 | 0.130 |
| | 932 | 947 | 0.110 |
| | 1266 | 1261 | 0.006 |
| | 800 | 826 | 0.373 |
| | 1188 | 1243 | 1.196 |
| | 1485 | 1466 | 0.111 |
| | 1241 | 1243 | 0.000 |
| | 1331 | 1300 | 0.334 |
| | 1589 | 1619 | 0.262 |
| | 1285 | 1215 | 1.932 |
| Repetition 2 | 1532 | 1515 | 0.082 |
| | 1144 | 1137 | 0.017 |
| | 1368 | 1294 | 2.048 |
| | 715 | 743 | 0.474 |
| | 883 | 910 | 0.352 |
| | 1043 | 1087 | 0.868 |
| | 818 | 809 | 0.039 |
| | 822 | 838 | 0.136 |
| | 1079 | 1065 | 0.079 |
| | 949 | 959 | 0.042 |
| Repetition 3 | 481 | 519 | 1.369 |
| | 735 | 759 | 0.354 |
| | 474 | 497 | 0.498 |
| | 405 | 426 | 0.481 |
| | 605 | 666 | 2.832 |
| | 707 | 733 | 0.434 |
| | 558 | 558 | 0.001 |
| | 673 | 733 | 2.476 |
| | 568 | 623 | 2.448 |
| | 896 | 860 | 0.698 |

Note:
^{★}represents that X²_{0.05} = 3.84 while df = 1.

It may be observed from the above embodiments 2 and 3 that the present disclosure realizes an disclosure purpose of stably creating rice male sterile line and maintainer line.

### Embodiment 4: Flanking Sequence Analysis of the Rice Event GSX2-55

Through performing detailed analysis on the DNA inserted into rice genome containing event GSX2-55 and genome sequence of the insertion fragment flanking, molecular features of the rice event GSX2-55 are analyzed and known. These analysis includes: an insertion fragment sequence, an insertion number (a number of integration sites in rice genome), a copy number (copy number of the insertion fragments in the gene locus) of insertion fragments, integrity of an inserted gene cassette and a flanking sequence of the inserted fragment and the like.

Tail-POR and DNA sequencing technology is used for determining 5′ and 3′ junctions of the inserted fragment and the rice genome, and determining a complete DNA sequence (SEQ ID NO: 9) of the inserted fragment in the rice containing the event GSX2-55.

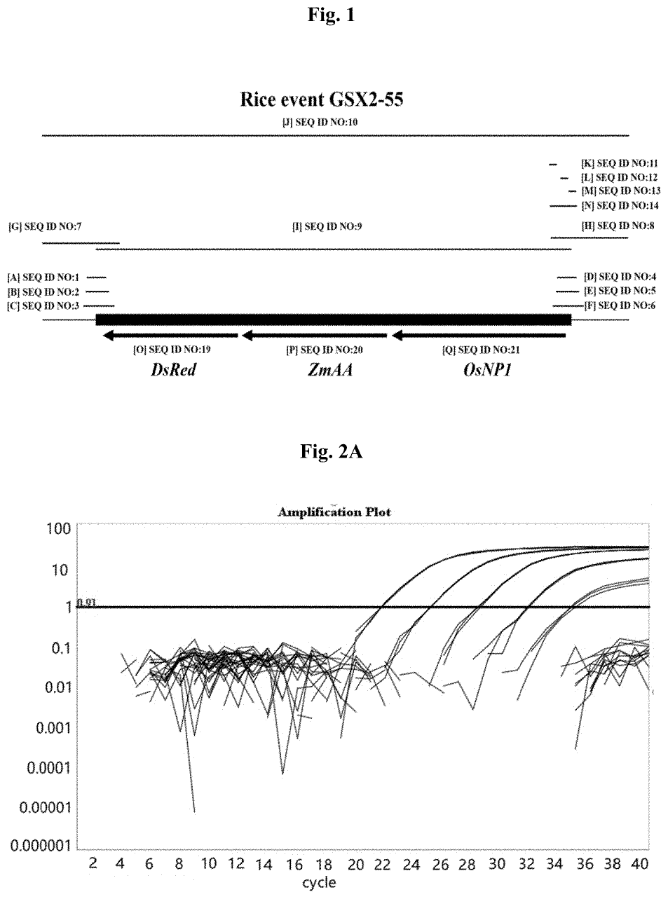

Flanking DNA sequence of the transgenic DNA sequence in the rice event GSX2-55 is cloned through HI-TAIL PCR (Liu et al., High-efficiency thermal asymmetric interlaced PCR for amplification of unknown flanking sequences (2007) BioTechniques 43:649-656) method. Operated in accordance with agricultural industry standards of the People's Republic of China NY/T674, the rice genomic DNA is extracted. The DNA is suitably diluted, an ultraviolet light absorption rate thereof in 260 nm and 280 nm is measured and recorded, and purified DNA concentration is calculated by an OD₂₆₀ value which is equivalent to 50 μg/mL of the DNA concentration. According to the measured concentration, the DNA solution is diluted to 50 ng/μL, and stored in 4 DEG C. and used within a week. Those skilled in the art may improve this type of the method so as to extract the genomic DNA from any tissues of the rice. According to the HI-TAIL PCR method, a primer combination is designed, and the genomic DNA is used as a template, according to a primer length and a specificity difference, an asymmetric temperature cycle is designed, and the TAIL-PCR is performed, an obtained PCR product is separated and purified through agarose gel electrophoresis, after that, a standard DNA sequencing scheme is used for sequencing the DNA product. A sequencing result is as shown in (FIG. 1), 5′ flanking sequence in the left border flanking sequence of the transgenic DNA is as shown in SEQ ID NO: 7; 3′ flanking sequence in the right border flanking sequence of the transgenic DNA is as shown in SEQ ID NO: 8; and the transgenic DNA part completely integrated in the rice genomic DNA is as shown in SEQ ID NO: 9.

After the flanking sequence of the transgenic DNA of the rice event GSX2-55 acquired by the HI-TAIL PCR and DNA sequencing is contrasted and analyzed with indica rice 9311 genome database (http://www.gramene.org/,ASM465v1), it is discovered that the rice event GSX2-55 exogenous sequence is inserted at the long-arm terminal of a rice genome chromosome 12.

### Embodiment 5: TAQMAN® Detection Method of the Rice Event GSX2-55

Extraction methods for genomes DNA of the rice event GSX2-55 and control samples (rice events FG2-47, FG2-205, FG2-525, FG2-670 and Zhen18A, corn Zheng 58, wheat ZhongguoChun, oilseed rape Zhongshuang 9 and soybean) are performed by referring to the embodiment 5. An ABI 7500FAST detection system is used for performing real-time PCR on each sample. A Taqman® probe TP55 (Applied Biosystems, Inc.) and primer combination TTY-55F/TTY-55R(SEQ ID NO: 11; SEQ ID NO: 12; SEQ ID NO: 13) is used for detecting the target sequence from the rice event GSX2-55. In addition, a second Taqman® probe and primer combination in allusion to rice endogenous sucrose phosphate synthase (SPS) gene is used for verifying that the amplified DNA (SEQ ID NO:15; SEQ ID NO: 16; SEQ ID NO:17; and SEQ ID NO: 18) exists in each real-time PCR reaction. The analysis verifies existence and/or inexistence of a real-time PCR qualitative positive and/or negative sample.

Positive and negative of the rice event GSX2-55 are determined on the basis of a Ct value of an event specificity target PCR, while a sample loading amount of the rice genomic DNA is 50 ng, an endogenous target and an event target are simultaneously amplified, and while the event target amplification Ct value is less than or equal to 36, a plant is assessed to be positive to the event; while the endogenous target and the event target are simultaneously amplified, and the event target amplification Ct value is greater than 36 and less than 40, the DNA is extracted again from the plant and real-time PCR analysis is repeatedly performed; if the endogenous target is amplified and the event target is not amplified (no amplification curve), the plant is assessed to be negative; if two targets are not amplified for a specific sample, it is determined that the sample is a DNA sample with poor quality or testing is failure, and the analysis is repeated.

It is shown from an experimental result that the rice event GSX2-55 is positive to an event specificity PCR result, but contrast rice (FG2-47, FG2-205, FG2-525, FG2-670 and Zhen18A), corn (Zheng 58), wheat (ZhongguoChun), oilseed rape (Zhongshuang 9) and soybean are negative to the event specificity PCR result (Table 3).

TABLE 3
specificity PCR analysis in the rice event GSX2-55 and contrast plants

| | | Rice endogenous |
|---|---|---|
| | Event specificity PCR | SPS gene |
| GSX2-55 | + | + |
| FG2-47 | − | + |
| FG2-205 | − | + |
| FG2-525 | − | + |
| FG2-670 | − | + |
| Zhen18A | − | + |
| Wuyun rice 7 | − | + |
| Corn (Zheng 58) | − | − |
| Oilseed rape | − | − |
| (Zhongshuang 9) | | |
| Wheat | − | − |
| (ZhongguoChun) | | |
| Soybean | − | − |

Note:
event specificity PCR measuring summary of the rice event GSX2-55, herein the positive(+) indicates that the rice event GSX2-55 is existent; and the negative(−) indicates that the rice event GSX2-55 is not existent.

### Embodiment 6: Sensitivity of TAQMAN® Detection Method of Rice Event GSX2-55

According to operating steps of plant genomic DNA extraction kit (DP350) of the TIANGEN BIOTECHBeijing Co., Ltd, genomic DNA of the rice event GSX2-55 and transformation acceptor Zhen18A is extracted, the genomic DNA of the rice event GSX2-55 is quantified to 10 ng/μL, 10 ng/μL of the Zhen18A genomic DNA is used as a diluent for continuously diluting the genomic DNA of the rice event GSX2-55 according to 10 times of a gradient (100%, 10%, 1%, 0.1%, 0.01%, 0.001%, 0.0001% and 0.00001%), and the diluent is used as a template for performing specificity PCR amplification (setting 3 parallels in each concentration gradient, and testing is repeated for 3 times), and used for detecting a lowest limit of detection (LOD) of the system (Table 4).

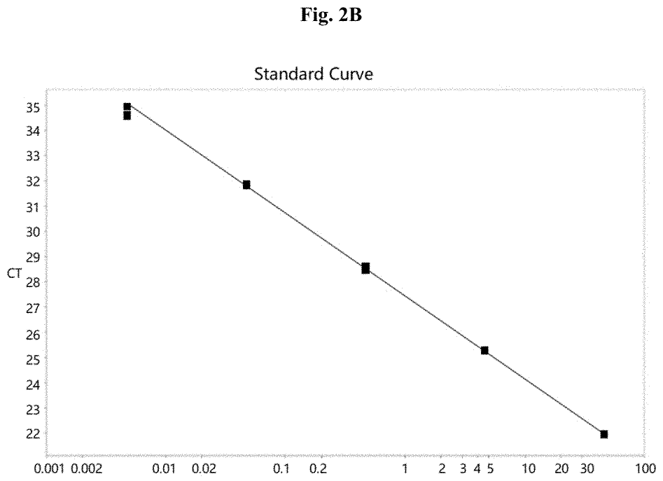

Through detecting the DNA of the rice event GSX2-55 in 10 times of the gradient, the quantitative PCR amplification system may effectively detect the DNA of the rice event GSX2-55 in different concentration, and shows a good linear relation (FIG. 2), a linear equation is Y=−3.25*Lg X+27.372; a coefficient of determination is R²=0.999; and quantitative PCR system amplification efficiency is 103.096%.

While a sample loading amount of the rice event GSX2-55 is 0.005 ng (0.01%), there is an apparent amplification curve, and a detection result may be judged to be positive. In other words, in a rice genomic DNA sample of which the sample loading amount is 50 ng, the quantitative PCR may specifically detect the DNA sample of which genomic DNA content of the rice event GSX2-55 is 0.01%.

TABLE 4
Standard curve Ct value of quantitative PCR system amplification

| GSX2-55 DNA | | | | |
|---|---|---|---|---|
| sample loading | | | | |
| amount (ng) | Ct value 1 | Ct value 2 | Ct value 3 | Ct mean |
| 50 | 21.7481 | 21.77558 | 21.77645 | 21.76671 |
| 5 | 25.12457 | 25.09439 | 25.12196 | 25.11364 |
| 0.5 | 28.38204 | 28.35718 | 28.49834 | 28.41252 |
| 0.05 | 31.8074 | 31.75273 | 31.74871 | 31.76962 |
| 0.005 | 34.54358 | 34.92086 | 34.60136 | 34.6886 |

What is claimed is:

1. A recombinant DNA molecule comprising:
a) a heterologous nucleic acid molecule inserted into rice genomic DNA and flanking rice genomic sequences thereof, and junction regions of the heterologous nucleic acid molecule to the 5′-end flanking sequence and 3′-end flanking sequence of the rice genome, wherein the heterologous nucleic acid molecule, the flanking sequences thereof and the junction regions are as shown in SEQ ID NO: 10, wherein partial sequence of SEQ ID NO: 10 is SEQ ID NOs: 1-9; the junction region at the 5′ end is as shown in SEQ ID NO: 1, 2, 3, or 7, and the junction region at the 3′ end is as shown in the sequence of 4, 5, 6, or 8; or
b) sequences complementary to (a).

2. The recombinant DNA molecule of claim 1, wherein the heterologous nucleic acid molecule comprises DsRed/ZmAA/OsNP1 gene, and has a sequence as shown in SEQ ID NO: 9.

3. The recombinant DNA molecule according to claim 1, wherein the recombinant DNA molecule is derived from transgenic rice plants containing event GSX2-55.

4. The recombinant DNA molecule according to claim 1, wherein the recombinant DNA molecule is comprised in rice plants, plant cells, seeds, and plant parts.

5. The recombinant DNA molecule according to claim 4, wherein the rice seeds produce red fluorescence when excited.

6. The recombinant DNA molecule according to claim 2, wherein the recombinant DNA molecules are amplicons generated from template molecules of event GSX2-55.

7. A DNA probe or primer pair for identifying the presence of event GSX2-55 in a biological sample, wherein the DNA probe or primer pair is designed according to the sequence of SEQ ID NO: 10 and used to identify the presence of the nucleotide sequences of SEQ ID NOs: 1-10 or complementary sequences thereof.

8. The DNA probe or primer pair according to claim 7, wherein the probe or primer has at least 10 nucleotides in length.

9. The DNA probe or primer pair according to claim 8, wherein the probe or primer has at least 18 nucleotides in length.

10. The DNA probe or primer pair according to claim 9, wherein the probe or primer has at least 24 nucleotides in length.

11. A DNA detection kit, characterized by comprising the DNA probe or primer pair for identifying the presence of event GSX2-55 in a biological sample of claim 7.

12. A method for detecting presence of DNA molecule of event GSX2-55 in a biological sample, characterized by comprising the following steps:
a) extracting DNA samples from a biological sample;
b) detecting the DNA samples by using the DNA probe or primer pair according to claim 7; and
c) determining that the event GSX2-55 or progeny thereof is present in the biological sample, if the presence of a nucleotide sequence comprising any one of SEQ ID NOs: 1-10 is detected.

13. A method for producing rice seeds, characterized by comprising the following steps:
a) planting rice seeds containing event GSX2-55 in the field, the DNA molecule of event GSX2-55 is the recombinant DNA molecule of claim 1;
b) performing selfing of rice comprising the event GSX2-55, or performing sexual crossing of rice containing the event GSX2-55 as a male and second rice plants;
c) harvesting rice inbred or hybrid seeds; and
d) selecting progeny plants with non-red fluorescent seeds in the excited state,
wherein the second rice plants contain osnp1/osnp1 gene that is mutated and loses fertility.


SEQUENCE LISTING (21 sequences)

SEQ ID NO: 1 - DNA, 20 bp, Artificial Sequence
  recombinant DNA molecule
tgaatgatggtgtaaacaaa

SEQ ID NO: 2 - DNA, 60 bp, Artificial Sequence
  recombinant DNA molecule
acaactatttaagtcgcatgtgaatgatggtgtaaacaaattgacgcttagacaacttaa

SEQ ID NO: 3 - DNA, 100 bp, Artificial Sequence
  recombinant DNA molecule
tatttttttaaaatttttctacaactatttaagtcgcatgtgaatgatggtgtaaacaaattgacgcttagacaacttaataacacattgcggacgtttt

SEQ ID NO: 4 - DNA, 20 bp, Artificial Sequence
  recombinant DNA molecule
cagtgtttgagtgtagaaaa

SEQ ID NO: 5 - DNA, 60 bp, Artificial Sequence
  recombinant DNA molecule
ttcaattgagtttaaactatcagtgtttgagtgtagaaaatccttatccgttctcaccat

SEQ ID NO: 6 - DNA, 100 bp, Artificial Sequence
  recombinant DNA molecule
gagtgacaaatggtaaattcttcaattgagtttaaactatcagtgtttgagtgtagaaaatccttatccgttctcaccatatctggcttattcctcaaat

SEQ ID NO: 7 - DNA, 1386 bp, Artificial Sequence
  recombinant DNA molecule
tgagctttaggagttaggggacattaactttatactaagataggattaacacatatcttcttaaattatcagtacgttatattttcccagaattcctgctatatatggacactactcttaaatgttacaaattaattcttgatcgagtaaattgcactcatcgtgaatgagtttacgggtggatacaaatagatgcaataacatacaaaacacttagtattactctgcaaacttggttagtccaaacttggttaattaaaaccggccactaccagcgaaatgattaattagatgacgctagctttcttatgattccaagattccaacccatgctagcaggatagtacaaatattctaaaaaaaagcaggatagtacaaattgttctttttctgcagaacccgtaacttgtagctttatatttaaggtccctatattttccagtggctaattttgctttgcaaatttactccacgctagtagttccagtgtggttccagcggatgaggctatccttgctttatgcaattttcagttgtgtgccactaacaaatcttttcactccaattagttcctaccacggaaagtattttctacatctaggtgccactttcttacacctaggtaccatggcacctaggtgtaggtaccatggcacctaggtgtatgcatgtaacttaaacagttatataaaaatttcataaaatttgataagatagcctatgataatataagacactacacaaacatacaagtctaaattcgatcaatatatgaagacacaaaaataacaaattttatctgcactatacgggcactattcagtgtttaattttattatttttgtttctctagttgtagatcaaatttagtcctgcatgtttgtgtatatacttatatcatcacaaacaatcttactatttttttaaaatttttctacaactatttaagtcgcatgtgaatgatggtgtaaacaaattgacgcttagacaacttaataacacattgcggacgtttttaatgtactgaattaacgccgaattggccgcattcgcaaaacacacctagactagatttgttttgctaacccaattgatattaattatatatgattaatatttatatgtatatggatttggttaatgaaatgcatctggttcatcaaagaattataaagacacgtgacattcatttaggataagaaatatggatgatctctttctcttttattcagataactagtaattacacataacacacaactttgatgcccacattatagtgattagcatgtcactatgtgtgcatccttttatttcatacattaattaagttggccaatccagaagatggacaagtctaggttaaccatgtggtacctacgcgtt

SEQ ID NO: 8 - DNA, 1440 bp, Artificial Sequence
  recombinant DNA molecule
agtggtggctagggcttgactatttgtttgtattggagggtggagataaaatgtagattttagatgacagatcttgcgattgagaatttgaaaaatctggtgggggattttcttactaatattacttggggttatttggttagttgcttcactttgtaatgccacattttgattatggcgtagttttctaggtaaatgtttgcttcgcccccacacctttttagccttaggacatagtatatgttatactactacctccgtttcaaaatatattttagtccaatttatagtattgggctgagtttagtgtcatagaaaatagggggagaatttactatttgcaactaccaaaatgatagcactcaaataccactctcctaaattttcattcccaaatgccatccgaacgcacatgtcagcctcacttttcattcaaacataagtggtgaggtccgggtgacatttaggaatgaatatttgggagagtggcatttgagcactagtattttggtagagtgacaaatggtaaattcttcaattgagtttaaactatcagtgtttgagtgtagaaaatccttatccgttctcaccatatctggcttattcctcaaataagccaaacggtatatttgcataaaaaataatttatgaataaaacttttatatgtgtgttcttagcgatttgaaaacaaaggctgaaaaaaataaacttcgttaaaaaaatcccaaaaccaaactccaaatttaatttttggctgataagtccaaaatctagtggttacaagagccttagtagcaccttagattctgggttcgcctccccgtggaagcgaattttccaggatttaacggctttgtgctttcagtggtaggcaacgtaaccgtcgacaacgaggcgcccgtggtgactttgtcaatctccaagatggatttgccagcacagtcttcgaagatgctcataggggtagggtttacgtgtatgcgttcataggtgtgaggatgctcataggggtagggtttacgtgcgtgcgttcataagggtgagtatgtgttgtgagtgtttgtgttgtactgtgtaattcttaaagaaaaaaacttcaaatttaaattttggctgataagtccataaacataagcatagtcagcctgctaaccttcttctgtatccatgcggcgtagcgcgtgaaatcttttttagtagctctgaaacaaacaaatgcagccacgagattagggatggcaacggggcggggcagggccaggttggtcaagaatgcccccgcccccgctcccgttccctgactctctcccccgttcccgcccccgaagcccagatcgggtgcaaaacaagacctgcccccgcacaccttccgggtccccgcacccccgggttagtagcggcgccgaccagtaaaatatggttgtttctcctctacaggc

SEQ ID NO: 9 - DNA, 12598 bp, Artificial Sequence
  transgenic expression cassette sequence
tgtaaacaaattgacgcttagacaacttaataacacattgcggacgtttttaatgtactgaattaacgccgaattggccgcattcgcaaaacacacctagactagatttgttttgctaacccaattgatattaattatatatgattaatatttatatgtatatggatttggttaatgaaatgcatctggttcatcaaagaattataaagacacgtgacattcatttaggataagaaatatggatgatctctttctcttttattcagataactagtaattacacataacacacaactttgatgcccacattatagtgattagcatgtcactatgtgtgcatccttttatttcatacattaattaagttggccaatccagaagatggacaagtctaggttaaccatgtggtacctacgcgttcgaactacaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtggccacgccgccgtcctcgaagttcatcacgcgctcccacttgaagccctcggggaaggacagcttcttgtagtcggggatgtcggcggggtgcttaacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatcacgttctcggaggaggccattactcggctacactcacacgctcgctctcgcagttgcaggtgtaagtttctagctagggcactcacggggtacgtatttgtagccagccacgcacggtctgagctcgccatgtgccgccatgcatgcgggggcacgtcgccagcgtacgcggccatcgtcgctgacgaaggtagcgcattcaagtccggtcggtagaggtcagctgggtcgttcgccgatggtagttgccgcccggactcagtgggcggtaggcgaaggctagcaagcagacgactccattcatgcgcatcatccaaaggtgatgcaaagccttccaaacgcgattgtctcatgatgtttccgtctcttgttacgaggagtacaattttttcttatacacgaacgttactttatgtcacatttccatgccatgaacaccttggcttcaaataagtgagtgttttttttcacattctgtggcataaacagaatttctagagtggcatttgtgatacattgtgaaagctaagagtggtaaaagtaaaataaaattgttttgcttttgccgcggaatggaaattatttgtcaaaacctaagagtggcaaaactgaaatgtcaaaacctagagtgacataaacaaaatttacccatcactaaatgagcacaaaatatttcaccacaatggaggtatgtgaggtccgatgtactactagagctcatcggaaaagcatcctcttgatgagtaaacctcttgaagtactgtaccaccacattttatttatcctcatcggcttatttttaggccacggttattctcacgaagagacggttggcgcgccatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagcgtgtcgtgctccaccatgttgacgggcgcgccaattcgagctcggtacccggccgctctagaactagtggatcccccgtccgatatcccgggtccttaggaagaccagatcctcaggatttggccgcgtcgacgtggagatataggggaaagagaacgctgatgtgacaagtgagtgagatatagggggagaaatttagggggaacgccgaacacagtctaaagtagcttgggacccaaagcactctgttcgggggtttttttttttgtctttcaactttttgctgtaatgttattcaaaataagaaaagcacttggcatggctaagaaatagagttcaacaactgaacagtacagtgtattatcaatggcataaaaaacaacccttacagcattgccgtattttattgatcaaacattcaactcaacactgacgagtggtcttccaccgatcaacggactaatgctgctttgtcagatctcaaggaaaagacgttatgcagtgtcgtgctgctgtgtttgcaatgcagcacaagactgagcaatctgagctgctataggtggcgccccgccgggacgcggaggctcgctttctcccagacgcagtagtccttgccgtgcgccgccgggtggaaatccgacgggaccacgctgctcacgccgtaccttgtcccgatcttcaccatgaccttctcgtcgacgacggccacgtacgcgtccgcgtccgccacgaggatccgcagcttgctcccggcgcggatgccgttccgcgccctgatggcagacagcgtggatatctcctgcttcaggttccagtcgaacatgtggtcgtagaaaatgcaggggactcctggatgggtgaggatgtaggcgtagccctgcatgaccttgtctgatgggaacggccagagcttctgcgtcgacccggtgtcatggttgtcgacgaaggtgacggccttctcgggcgcccacccgatcaggccggccgcgttgccggagctgtcgcgcagccgccacagctccccctgcacgcccgcctgcagcaggcccttggtggggaagtcgaacgccatggcgggcccgccgacggcccgcgtccagtccagcagctcctgccggcactggtcctggttgggcgccggcttgccgtccccgctgtagctcagcgagttccatatctccgcgacgacgaagctcggcggccccgtgctctccacgtacattctggcgacggccggcgagtagcccttggcgaagtcgaggcgccagccgtcgaaccccacggcgtcggacctgagccagttgagccaggcggagagctcccgctgcacgcgcgggttgaggtggtcgatgtcgggcgccgccgcgaacccctcgcccgtgtcgcggtgccccgtcccgtccgagtactgcgtgtcgtcgctgcagatcatcccggggccccagtccaggcggtcgtcgggagtcccgccctcgaagatgcagtacacgccgcgcgcgtccttcttttccgcgcaccggtggttgatgacgatgtccgccacgcactgcacgcccctgccgtggaacgccgctatcagggacttgagctccgccgccgtgccgtacttggacgcgtccaggtcgtataggcggcctggcatgtagccttgtggcgagacggagtgcgagggtggaggcagccagacgtgcgtgacgccggccttggcgatgtcgtcgacctgggccttgagcctgttgtaccagcctccctgctgcttgcacgactcccagttaaacccctggaagaggacttgtgcctggaccaggccgcaggccgcccggacgaccttgggatcgggctcccgcgggtcgcgccccgccgggacgcccgggcacacgttgaggccgacgctggcgaacatcgcccggcgagggaactcgaggccgccggtcttgccacctcgtggcttccaagggaatgccaccgccgggacctgcaatgacgaccagctctccttgctcctcgtcaccatcgtcgtcactgccattgtcgccgccatggtgtcgtgatcgatgctttattcgtgtctcttgttgcctgggcactaggacctataaataccattgttctgctgataaaattagtgcgctatatgtatggcttggacaccatgcctttgcatcgctatttttagggcagacttcttgtcctcaaactcttcatgcattatttggacccttcagaagtaaccactaaccaccgtggaaagcataaattaaataacaaaagaaagaatgaacaatgccaacatttaaactatactctactatcttatatatatcttggtattactaattgaaggttctaatagagcctctggattaattttcactctattattaattcaggacccaattgagcctttatgttaattctcatcagacatgataaaaaattaaaaaatatcataaattcttagattaattagaaatatctggccattaaacaagagactctaaattatacataactattagatcctgaaggaccaaaaaagtgatcaaatggggtgaataggtctatgttgagcaacctctcggctttgaagatagtgagtaccctaaccatgtttataaactctcaaaggcgctttatgggctcaagcaagccccaacagcatggtatgaatgcctaagagattttcttatcactaatggcttcagagtcagtaaagccgatcctactctctttactaaaaccatttcaaaagttttgtttgtatgccaaatttatgttgatgatattatatttgggtctactaacaaatctacttgtgaagagtttagtaggatcatgattccgaaattcgagatgtctatgatggggaagttgaaatattttctaggatttcaagtcaagcaactccaagatggcaccttcatcagccaaacaaagtacattcaagacatactcaacaagtttggaatgaaggatgccaagcccatcaggacacccatgggaactaatgggcatctcgacctcgacacgggaggtaaatccgtagaccaaaaggtataccggtcgatgataggatctttactctatttatgtgcatctcgaccagatagtatgctttctatatgcatgtgtgcaagattccaagccgatcctaaggaagttcaccttagggccgtgaaaagaatcatgagatatttagtttacactcctaaatttggtctttggtaccccaagggatccacctttgatttaataggatattcagatgccgattgggcagggtgtaaaattgataggaagagcacatcagggacttgtcagtttctagggagatccctggtgtcttggacttcaaagaaacaaaactcaatagctctttctaccgccgaagccaagtatattgccgcaggacattgttgtgtgcaattactttggatgaggcaaaaccttagggactatggctacaaattgagcaaagtccctctcctatgtgacaatgagagtgctatctgcatggcggataatcccgttgaacacagccgcactaagcacatagacattcggtatcactttttgagggatcaccaacaaaggggtgatatcgagattgcttatgttagcaccaaagaacaattagtcgatatctttaccaaaccattagatgataaaacctttagcaaacttaggaatgagctaaatattcttgattctcgaaactttgattgaaacattacacacatagctcatttgtatacctttgatcatatctctttcgtggctacgactaatgtgttttcaagtgtatttctatgctaagtcgtagattgaaagggaaatggagtcttcggcgaagacaaggcttccactccactctaacggtatcgtttatccttcgccgtcactccgcatcactgtccaaatttggtataatctttcactcatatttcatttaccaatggggagaaagtataaatggctcacaaagtctccgtttttggcgattaatgccaaagggggagaaatattaagcccaaagcaaaaggaccgcaccaccactttttgaaattttttaaattggtatgtttaatttcaaattggtatgttgattttcaattggtatattttcaaaattagcatctaaatatatttccaattgatatctatttaaaaccctcttgaaagctaagaggagaattttattcagggggagttttgtttagtcaaaggaaaagcatttgaaacagggggagaaatttcaaatcttgaaaatgcttcttacaatcttattcatatacctttgactatttgcaaaagactttgaaaaagaatttccaaaaagatttgcaaaaaacaaaacaagtggtgcaaatgtggtccaaaatgttaaaataaaagaaagcaaccatgcatatcaagtaaaagtataaattgatttaattctaagtaacctatgcacttaccttatgcaaactagttcaattctgcacttatatattttctttggtttgtgttggcatcaatcaccaaaaagggggagattgaaagggaaataaggtttaaccttttcctataaataattttggtggttgaatgcccaacacaaatgattggactaactagtttgttctagattatatattccacaggtgcataaaggttcaacacaaaccaataaacgatcaaagttagggttcaaaagcaaaggagcaaaggaaccgaagggtgccctgatctggcacaccggactgtctggtatgccaccagacagtgtccggtgcagatcctctagagtcgacctgcaggcatgcaagcttaatccagccatatatatttgttgcaattatcttttgttttggcagagtaacctaaagggaaattcgtaccctatcaatcatatttttttagataaggctaaaaaaaagctaccactcactcttaagaatatgttaattggaagaaaaaaagagaagctaccattcattctcaagaatatgttaattgaagaaaaagagagctatctacaccatccatcatggtgggaccagattgtagctacaaatgtatggaaaatacaaatgtatttgtagaaatagagaaaaagttgcatatcaagtacagtcatatttcaaatagccctgtacaactgtgaaggaatctcgactagttgtctcttggtttgagggatatacctattttaggatgactgatatttctagaaccacactctaattcaaatctgctgatttcatgtgtagatttatgggtacagaatgtctctttgggcggagcatattggagtggttgaggaaggcttcaactatccagagaccatggaatgcatgagacgagttcgccaaatcggtgaacagaactgggaacgatttgttgataatgaggtgactgagatgagaggccacctaatgaagtaccctgtaagtgttgaccgtaagggcaaggtgaaacccttgccaggatgtacgtcattcccagacatgggtggtaacatctgtggatcattccgagccatccaagaaaacctcacaatatgaatttcgattatctgtaagtatacaattgctagtgattctgagctattctgcttcctgaggttatagatagcgtgaaaattttgctagcaatataggttgaaaccaagtaggacatgacaagaaaaggttattaccctgcattttttatgcctttcttaagagaattcaacatttccatcttaataggttttctttttatgtatactttttatcaatgtatagaagattaagaatgtttgaaagcccggaaccctctgtttagggaacatggctttggtagcaactttgattgatttaagtatgtagcgacatgtaaagccattaaaataagtttctagtgtaattattttcagccttgatttgctggttccattttctaagtgtcaatgaaatctaatccatttatttcttcgtcatgcccttggtaacataagtttccgctttgtttttgcaatgcttcttcaaggttacataatcaacaagcagatactgaacctatgtctagagaagagtaaaaattaagtgggtgcaaataaaatctactctaaactctaaagacagttgaacattttcttttccttgaattacatgcatttttttgctcctgaaacgaaattatcttttgttcatcatttcttccatctctcggactgaatcttcacacccatatacctgcccagtgtccatattgaaaactatactgttagtgtgttgttactcatggcaatgcactttcatgagctaaaatgatgccagttacctgccgagcatcatgacggtggcctgagggttggtgccgggggagtacttgaaggtggagctgtcgatcaccctgagcccctgcaccccgaacacccggtaatcgtcgtcgacgacggcgccgacatggcagccgccgtggtagtgccagatggtcatgacggtgtccatgcagtactgctccggcgagcgcgagtcgttgacatggcgcggcagcaggttgacggggaagttggcggaatcggtgaagatggactcgacggaggcgttggcgtaggtgaagttggagaaggcccgcgactggatcacccgctcgatcgtctcgatgccatggacgcaccgctccagatcctctgcctcgcggaagtagttgaacgtcaccgacgggttcgccctcgggtcggtggttcgcagctcgacgtggccggaggacaccggcccgaggatcttctccaggatgaagcctccccggaacgccctcctgtccagccgcatcatcgcctccgccgcccgctgcagcgcctccggcgtcctctgcttcggcggcagcgtgccgagctgccccgtctgcggcgacagcatcccgaagctgcgcgcccaccggagcgcgccgtccgacaccggcatgccgaactccgacccgctcaccccctcgatgaagctgccggagcgggtgatgccgacgacctgcaccagggagagctccaccggcaccggcgacgggatgaacaccgagttcatcgggttgtcggcgacgccctgcccgaccatgggttggtccacgatcacctcgatgccgtgcgcctccaggtgcgcctgcggcccgacgccgctcagcatcagcagctgcgggctccccagcgtccccgccgacacgatcacctcgttcttgtcgccgtcgcggaggtacacccggtgctgcacccccagcgggtccgagaacaccaccccgtacgccaccgggtatgggacgccgtctgcacgccatggcaaaagctcgccgtcgtgaggtcaagcttcttcggcgcacagcacattttccattttcatcgtagctgtgtaccttggcttttgaagaggatacgggagacggtggcgtagaggaggacggtgaggccgcgggggcgggcgtggcggaggaagtcggcggcggtgtggcgctggccggagttgtcgaagatggtgccgccgatcttggtgccggtgacgtggtcgaaggtgaagccgttgtcgggcgtgacgccgacctcgagcagcgcgtcgcggagcgccgcctgccacggcggcacgtcggggcggaacaccagcgagcgctccacccaccggtacgacgagttcaccagccgcgcgtcccacccggaggcgcgcacgtactcgttgctcgcccgcgtgtagaacccggcgttgaggcagctcccgccgccgagcacccgcgcccgggcgttcaccacgccgtcctccgagatgaaccgctgcgccggcgacgccggcgacgtgtcggccagcgcgtccgtgaagtgctgctcgctcgacatgttggcgtacggcaggccgccgcgctccagcagcagcacgcgcgagtgctccgacagcgtcgccgccagcgggcaccccgccgtgccgccgccgatgacgatgtagttgtagctcgacaccagcggcgcgtcctgcgcgtgccgcacgaaccgcaggttctccagttgctctgcacaacgccatcgcacgccatgaacacgacgacgacgcaacacgcatcaacgcaaagatacaacgcaacgtttacgcaccttgctcttccgagagcgcggcgaggcagagcgagaggagcacggcggcggcggcggcaagcaccggcgccgacgagctcgcgcggccaagtgctgccattttcgccgggcaaattcgctaactttcacattcccctgggacgaaacgttcggtctgtttatatatccggagttagctcaatggtagatcaagagtagatgtcttgaataatcttgcagattgctttgcactgcaaagaagatcttcgagcagctttgtaaattgtggagccctgagaagaatataacatgcatgcagaagtgatgagctgatgaaacattgatcaatgatctgatgtggttgtaagcaagcaagcaggagagtttacttgccaatcttgcgattgtttcgaacgtgctttgatatttttgccattttaaaatcataatttagtcgttgctgagttatttttctgttacgagacagtcggggcaggctggatttcaatggtttcttcttgatttattaagtcgtaaaagaagcgtaattttaatgagttgggtcagctcttgggcaagattatgacggtgcttaccatgtatgcatctcaactaaatctgataaagccccattgaaacggtccatttgtacaaaaaaatattaaaatattaggtgatttaattttagagtaaattttactatagacgaacttttattagcgatgtttatcaatgttctcactttataccggttattattcttacccatcctcaactcttttatccatatataacttgtcatataccgatgaatggttttacatatatatgtgagagtttattgatgagtaggagccaatgtaatcgagattgttcatgctcgagaagagagatgggtggtatgtggtccaaagtgaagcatctgtaacaaaaggtggtccaaatgaatttgctcttaactttaaattaacttcttggcccattgcagttgtattgcccaatggtaaagttgtaagttttaagacggttggtttctttttttgtgttttactcctcctccttctttcttcgtcttcggattgctatacgccagtctagtaaaaaaaatatccatttccctaaaccacattcgattgaaaattaagaggaaaatcttacaaatatctaacctttatatctacctcacaatttctgctctctgcctctccaacaatgttgattatgtccagagacattgacagtggtctgaccagttagagagagcataggttcctccgcttagagtgaggctaaggagatagcggcctgatggtggacggtggcattgggaaggcaacgagatgacagtgacgccgtcgtagctacgaggacaatggatagtggttatggcgagagaatcccgatcatgtaattaggtgatggtgagttgctcgagacggaagatgagaatagggtcaggcacctcgctagattggttaggtggtggtggtcatgaattcagcgacaagaccattagagacgaggaagacaacagcgtaaggacataagaaatggtgatggcacatgagaagccatctcaaggaggatgatggcaatagggatggcgatggaggtgcttgtcggcgagcatcaccagcgttgagggagatgccattgaggctaaatggtggtggtgcagagggcatgattggttcagcgtagcacagggtgaggttaggaggagcgatgtggaagtggtggctagggcttgactatttgtttgtattggagggtggagataaaatgtagattttagatgacagatcttgcgattgagaatttgaaaaatctggtgggggattttcttactaatattacttggggttatttggttagttgcttcactttgtaatgccacattttgattatggcgtagttttctaggtaaatgtttgcttcgcccccacacctttttagccttaggacatagtatatgttatactactacctccgtttcaaaatatattttagtccaatttatagtattgggctgagtttagtgtcatagaaaatagggggagaatttactatttgcaactaccaaaatgatagcactcaaataccactctcctaaattttcattcccaaatgccatccgaacgcacatgtcagcctcacttttcattcaaacataagtggtgaggtccgggtgacatttaggaatgaatatttgggagagtggcatttgagcactagtattttggtagagtgacaaatggtaaattcttcaattgagtttaaactatcagtgtttga

SEQ ID NO: 10 - DNA, 14441 bp, Artificial Sequence
  recombinant DNA molecule
tgagctttaggagttaggggacattaactttatactaagataggattaacacatatcttcttaaattatcagtacgttatattttcccagaattcctgctatatatggacactactcttaaatgttacaaattaattcttgatcgagtaaattgcactcatcgtgaatgagtttacgggtggatacaaatagatgcaataacatacaaaacacttagtattactctgcaaacttggttagtccaaacttggttaattaaaaccggccactaccagcgaaatgattaattagatgacgctagctttcttatgattccaagattccaacccatgctagcaggatagtacaaatattctaaaaaaaagcaggatagtacaaattgttctttttctgcagaacccgtaacttgtagctttatatttaaggtccctatattttccagtggctaattttgctttgcaaatttactccacgctagtagttccagtgtggttccagcggatgaggctatccttgctttatgcaattttcagttgtgtgccactaacaaatcttttcactccaattagttcctaccacggaaagtattttctacatctaggtgccactttcttacacctaggtaccatggcacctaggtgtaggtaccatggcacctaggtgtatgcatgtaacttaaacagttatataaaaatttcataaaatttgataagatagcctatgataatataagacactacacaaacatacaagtctaaattcgatcaatatatgaagacacaaaaataacaaattttatctgcactatacgggcactattcagtgtttaattttattatttttgtttctctagttgtagatcaaatttagtcctgcatgtttgtgtatatacttatatcatcacaaacaatcttactatttttttaaaatttttctacaactatttaagtcgcatgtgaatgatggtgtaaacaaattgacgcttagacaacttaataacacattgcggacgtttttaatgtactgaattaacgccgaattggccgcattcgcaaaacacacctagactagatttgttttgctaacccaattgatattaattatatatgattaatatttatatgtatatggatttggttaatgaaatgcatctggttcatcaaagaattataaagacacgtgacattcatttaggataagaaatatggatgatctctttctcttttattcagataactagtaattacacataacacacaactttgatgcccacattatagtgattagcatgtcactatgtgtgcatccttttatttcatacattaattaagttggccaatccagaagatggacaagtctaggttaaccatgtggtacctacgcgttcgaactacaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtggccacgccgccgtcctcgaagttcatcacgcgctcccacttgaagccctcggggaaggacagcttcttgtagtcggggatgtcggcggggtgcttaacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatcacgttctcggaggaggccattactcggctacactcacacgctcgctctcgcagttgcaggtgtaagtttctagctagggcactcacggggtacgtatttgtagccagccacgcacggtctgagctcgccatgtgccgccatgcatgcgggggcacgtcgccagcgtacgcggccatcgtcgctgacgaaggtagcgcattcaagtccggtcggtagaggtcagctgggtcgttcgccgatggtagttgccgcccggactcagtgggcggtaggcgaaggctagcaagcagacgactccattcatgcgcatcatccaaaggtgatgcaaagccttccaaacgcgattgtctcatgatgtttccgtctcttgttacgaggagtacaattttttcttatacacgaacgttactttatgtcacatttccatgccatgaacaccttggcttcaaataagtgagtgttttttttcacattctgtggcataaacagaatttctagagtggcatttgtgatacattgtgaaagctaagagtggtaaaagtaaaataaaattgttttgcttttgccgcggaatggaaattatttgtcaaaacctaagagtggcaaaactgaaatgtcaaaacctagagtgacataaacaaaatttacccatcactaaatgagcacaaaatatttcaccacaatggaggtatgtgaggtccgatgtactactagagctcatcggaaaagcatcctcttgatgagtaaacctcttgaagtactgtaccaccacattttatttatcctcatcggcttatttttaggccacggttattctcacgaagagacggttggcgcgccatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagcgtgtcgtgctccaccatgttgacgggcgcgccaattcgagctcggtacccggccgctctagaactagtggatcccccgtccgatatcccgggtccttaggaagaccagatcctcaggatttggccgcgtcgacgtggagatataggggaaagagaacgctgatgtgacaagtgagtgagatatagggggagaaatttagggggaacgccgaacacagtctaaagtagcttgggacccaaagcactctgttcgggggtttttttttttgtctttcaactttttgctgtaatgttattcaaaataagaaaagcacttggcatggctaagaaatagagttcaacaactgaacagtacagtgtattatcaatggcataaaaaacaacccttacagcattgccgtattttattgatcaaacattcaactcaacactgacgagtggtcttccaccgatcaacggactaatgctgctttgtcagatctcaaggaaaagacgttatgcagtgtcgtgctgctgtgtttgcaatgcagcacaagactgagcaatctgagctgctataggtggcgccccgccgggacgcggaggctcgctttctcccagacgcagtagtccttgccgtgcgccgccgggtggaaatccgacgggaccacgctgctcacgccgtaccttgtcccgatcttcaccatgaccttctcgtcgacgacggccacgtacgcgtccgcgtccgccacgaggatccgcagcttgctcccggcgcggatgccgttccgcgccctgatggcagacagcgtggatatctcctgcttcaggttccagtcgaacatgtggtcgtagaaaatgcaggggactcctggatgggtgaggatgtaggcgtagccctgcatgaccttgtctgatgggaacggccagagcttctgcgtcgacccggtgtcatggttgtcgacgaaggtgacggccttctcgggcgcccacccgatcaggccggccgcgttgccggagctgtcgcgcagccgccacagctccccctgcacgcccgcctgcagcaggcccttggtggggaagtcgaacgccatggcgggcccgccgacggcccgcgtccagtccagcagctcctgccggcactggtcctggttgggcgccggcttgccgtccccgctgtagctcagcgagttccatatctccgcgacgacgaagctcggcggccccgtgctctccacgtacattctggcgacggccggcgagtagcccttggcgaagtcgaggcgccagccgtcgaaccccacggcgtcggacctgagccagttgagccaggcggagagctcccgctgcacgcgcgggttgaggtggtcgatgtcgggcgccgccgcgaacccctcgcccgtgtcgcggtgccccgtcccgtccgagtactgcgtgtcgtcgctgcagatcatcccggggccccagtccaggcggtcgtcgggagtcccgccctcgaagatgcagtacacgccgcgcgcgtccttcttttccgcgcaccggtggttgatgacgatgtccgccacgcactgcacgcccctgccgtggaacgccgctatcagggacttgagctccgccgccgtgccgtacttggacgcgtccaggtcgtataggcggcctggcatgtagccttgtggcgagacggagtgcgagggtggaggcagccagacgtgcgtgacgccggccttggcgatgtcgtcgacctgggccttgagcctgttgtaccagcctccctgctgcttgcacgactcccagttaaacccctggaagaggacttgtgcctggaccaggccgcaggccgcccggacgaccttgggatcgggctcccgcgggtcgcgccccgccgggacgcccgggcacacgttgaggccgacgctggcgaacatcgcccggcgagggaactcgaggccgccggtcttgccacctcgtggcttccaagggaatgccaccgccgggacctgcaatgacgaccagctctccttgctcctcgtcaccatcgtcgtcactgccattgtcgccgccatggtgtcgtgatcgatgctttattcgtgtctcttgttgcctgggcactaggacctataaataccattgttctgctgataaaattagtgcgctatatgtatggcttggacaccatgcctttgcatcgctatttttagggcagacttcttgtcctcaaactcttcatgcattatttggacccttcagaagtaaccactaaccaccgtggaaagcataaattaaataacaaaagaaagaatgaacaatgccaacatttaaactatactctactatcttatatatatcttggtattactaattgaaggttctaatagagcctctggattaattttcactctattattaattcaggacccaattgagcctttatgttaattctcatcagacatgataaaaaattaaaaaatatcataaattcttagattaattagaaatatctggccattaaacaagagactctaaattatacataactattagatcctgaaggaccaaaaaagtgatcaaatggggtgaataggtctatgttgagcaacctctcggctttgaagatagtgagtaccctaaccatgtttataaactctcaaaggcgctttatgggctcaagcaagccccaacagcatggtatgaatgcctaagagattttcttatcactaatggcttcagagtcagtaaagccgatcctactctctttactaaaaccatttcaaaagttttgtttgtatgccaaatttatgttgatgatattatatttgggtctactaacaaatctacttgtgaagagtttagtaggatcatgattccgaaattcgagatgtctatgatggggaagttgaaatattttctaggatttcaagtcaagcaactccaagatggcaccttcatcagccaaacaaagtacattcaagacatactcaacaagtttggaatgaaggatgccaagcccatcaggacacccatgggaactaatgggcatctcgacctcgacacgggaggtaaatccgtagaccaaaaggtataccggtcgatgataggatctttactctatttatgtgcatctcgaccagatagtatgctttctatatgcatgtgtgcaagattccaagccgatcctaaggaagttcaccttagggccgtgaaaagaatcatgagatatttagtttacactcctaaatttggtctttggtaccccaagggatccacctttgatttaataggatattcagatgccgattgggcagggtgtaaaattgataggaagagcacatcagggacttgtcagtttctagggagatccctggtgtcttggacttcaaagaaacaaaactcaatagctctttctaccgccgaagccaagtatattgccgcaggacattgttgtgtgcaattactttggatgaggcaaaaccttagggactatggctacaaattgagcaaagtccctctcctatgtgacaatgagagtgctatctgcatggcggataatcccgttgaacacagccgcactaagcacatagacattcggtatcactttttgagggatcaccaacaaaggggtgatatcgagattgcttatgttagcaccaaagaacaattagtcgatatctttaccaaaccattagatgataaaacctttagcaaacttaggaatgagctaaatattcttgattctcgaaactttgattgaaacattacacacatagctcatttgtatacctttgatcatatctctttcgtggctacgactaatgtgttttcaagtgtatttctatgctaagtcgtagattgaaagggaaatggagtcttcggcgaagacaaggcttccactccactctaacggtatcgtttatccttcgccgtcactccgcatcactgtccaaatttggtataatctttcactcatatttcatttaccaatggggagaaagtataaatggctcacaaagtctccgtttttggcgattaatgccaaagggggagaaatattaagcccaaagcaaaaggaccgcaccaccactttttgaaattttttaaattggtatgtttaatttcaaattggtatgttgattttcaattggtatattttcaaaattagcatctaaatatatttccaattgatatctatttaaaaccctcttgaaagctaagaggagaattttattcagggggagttttgtttagtcaaaggaaaagcatttgaaacagggggagaaatttcaaatcttgaaaatgcttcttacaatcttattcatatacctttgactatttgcaaaagactttgaaaaagaatttccaaaaagatttgcaaaaaacaaaacaagtggtgcaaatgtggtccaaaatgttaaaataaaagaaagcaaccatgcatatcaagtaaaagtataaattgatttaattctaagtaacctatgcacttaccttatgcaaactagttcaattctgcacttatatattttctttggtttgtgttggcatcaatcaccaaaaagggggagattgaaagggaaataaggtttaaccttttcctataaataattttggtggttgaatgcccaacacaaatgattggactaactagtttgttctagattatatattccacaggtgcataaaggttcaacacaaaccaataaacgatcaaagttagggttcaaaagcaaaggagcaaaggaaccgaagggtgccctgatctggcacaccggactgtctggtatgccaccagacagtgtccggtgcagatcctctagagtcgacctgcaggcatgcaagcttaatccagccatatatatttgttgcaattatcttttgttttggcagagtaacctaaagggaaattcgtaccctatcaatcatatttttttagataaggctaaaaaaaagctaccactcactcttaagaatatgttaattggaagaaaaaaagagaagctaccattcattctcaagaatatgttaattgaagaaaaagagagctatctacaccatccatcatggtgggaccagattgtagctacaaatgtatggaaaatacaaatgtatttgtagaaatagagaaaaagttgcatatcaagtacagtcatatttcaaatagccctgtacaactgtgaaggaatctcgactagttgtctcttggtttgagggatatacctattttaggatgactgatatttctagaaccacactctaattcaaatctgctgatttcatgtgtagatttatgggtacagaatgtctctttgggcggagcatattggagtggttgaggaaggcttcaactatccagagaccatggaatgcatgagacgagttcgccaaatcggtgaacagaactgggaacgatttgttgataatgaggtgactgagatgagaggccacctaatgaagtaccctgtaagtgttgaccgtaagggcaaggtgaaacccttgccaggatgtacgtcattcccagacatgggtggtaacatctgtggatcattccgagccatccaagaaaacctcacaatatgaatttcgattatctgtaagtatacaattgctagtgattctgagctattctgcttcctgaggttatagatagcgtgaaaattttgctagcaatataggttgaaaccaagtaggacatgacaagaaaaggttattaccctgcattttttatgcctttcttaagagaattcaacatttccatcttaataggttttctttttatgtatactttttatcaatgtatagaagattaagaatgtttgaaagcccggaaccctctgtttagggaacatggctttggtagcaactttgattgatttaagtatgtagcgacatgtaaagccattaaaataagtttctagtgtaattattttcagccttgatttgctggttccattttctaagtgtcaatgaaatctaatccatttatttcttcgtcatgcccttggtaacataagtttccgctttgtttttgcaatgcttcttcaaggttacataatcaacaagcagatactgaacctatgtctagagaagagtaaaaattaagtgggtgcaaataaaatctactctaaactctaaagacagttgaacattttcttttccttgaattacatgcatttttttgctcctgaaacgaaattatcttttgttcatcatttcttccatctctcggactgaatcttcacacccatatacctgcccagtgtccatattgaaaactatactgttagtgtgttgttactcatggcaatgcactttcatgagctaaaatgatgccagttacctgccgagcatcatgacggtggcctgagggttggtgccgggggagtacttgaaggtggagctgtcgatcaccctgagcccctgcaccccgaacacccggtaatcgtcgtcgacgacggcgccgacatggcagccgccgtggtagtgccagatggtcatgacggtgtccatgcagtactgctccggcgagcgcgagtcgttgacatggcgcggcagcaggttgacggggaagttggcggaatcggtgaagatggactcgacggaggcgttggcgtaggtgaagttggagaaggcccgcgactggatcacccgctcgatcgtctcgatgccatggacgcaccgctccagatcctctgcctcgcggaagtagttgaacgtcaccgacgggttcgccctcgggtcggtggttcgcagctcgacgtggccggaggacaccggcccgaggatcttctccaggatgaagcctccccggaacgccctcctgtccagccgcatcatcgcctccgccgcccgctgcagcgcctccggcgtcctctgcttcggcggcagcgtgccgagctgccccgtctgcggcgacagcatcccgaagctgcgcgcccaccggagcgcgccgtccgacaccggcatgccgaactccgacccgctcaccccctcgatgaagctgccggagcgggtgatgccgacgacctgcaccagggagagctccaccggcaccggcgacgggatgaacaccgagttcatcgggttgtcggcgacgccctgcccgaccatgggttggtccacgatcacctcgatgccgtgcgcctccaggtgcgcctgcggcccgacgccgctcagcatcagcagctgcgggctccccagcgtccccgccgacacgatcacctcgttcttgtcgccgtcgcggaggtacacccggtgctgcacccccagcgggtccgagaacaccaccccgtacgccaccgggtatgggacgccgtctgcacgccatggcaaaagctcgccgtcgtgaggtcaagcttcttcggcgcacagcacattttccattttcatcgtagctgtgtaccttggcttttgaagaggatacgggagacggtggcgtagaggaggacggtgaggccgcgggggcgggcgtggcggaggaagtcggcggcggtgtggcgctggccggagttgtcgaagatggtgccgccgatcttggtgccggtgacgtggtcgaaggtgaagccgttgtcgggcgtgacgccgacctcgagcagcgcgtcgcggagcgccgcctgccacggcggcacgtcggggcggaacaccagcgagcgctccacccaccggtacgacgagttcaccagccgcgcgtcccacccggaggcgcgcacgtactcgttgctcgcccgcgtgtagaacccggcgttgaggcagctcccgccgccgagcacccgcgcccgggcgttcaccacgccgtcctccgagatgaaccgctgcgccggcgacgccggcgacgtgtcggccagcgcgtccgtgaagtgctgctcgctcgacatgttggcgtacggcaggccgccgcgctccagcagcagcacgcgcgagtgctccgacagcgtcgccgccagcgggcaccccgccgtgccgccgccgatgacgatgtagttgtagctcgacaccagcggcgcgtcctgcgcgtgccgcacgaaccgcaggttctccagttgctctgcacaacgccatcgcacgccatgaacacgacgacgacgcaacacgcatcaacgcaaagatacaacgcaacgtttacgcaccttgctcttccgagagcgcggcgaggcagagcgagaggagcacggcggcggcggcggcaagcaccggcgccgacgagctcgcgcggccaagtgctgccattttcgccgggcaaattcgctaactttcacattcccctgggacgaaacgttcggtctgtttatatatccggagttagctcaatggtagatcaagagtagatgtcttgaataatcttgcagattgctttgcactgcaaagaagatcttcgagcagctttgtaaattgtggagccctgagaagaatataacatgcatgcagaagtgatgagctgatgaaacattgatcaatgatctgatgtggttgtaagcaagcaagcaggagagtttacttgccaatcttgcgattgtttcgaacgtgctttgatatttttgccattttaaaatcataatttagtcgttgctgagttatttttctgttacgagacagtcggggcaggctggatttcaatggtttcttcttgatttattaagtcgtaaaagaagcgtaattttaatgagttgggtcagctcttgggcaagattatgacggtgcttaccatgtatgcatctcaactaaatctgataaagccccattgaaacggtccatttgtacaaaaaaatattaaaatattaggtgatttaattttagagtaaattttactatagacgaacttttattagcgatgtttatcaatgttctcactttataccggttattattcttacccatcctcaactcttttatccatatataacttgtcatataccgatgaatggttttacatatatatgtgagagtttattgatgagtaggagccaatgtaatcgagattgttcatgctcgagaagagagatgggtggtatgtggtccaaagtgaagcatctgtaacaaaaggtggtccaaatgaatttgctcttaactttaaattaacttcttggcccattgcagttgtattgcccaatggtaaagttgtaagttttaagacggttggtttctttttttgtgttttactcctcctccttctttcttcgtcttcggattgctatacgccagtctagtaaaaaaaatatccatttccctaaaccacattcgattgaaaattaagaggaaaatcttacaaatatctaacctttatatctacctcacaatttctgctctctgcctctccaacaatgttgattatgtccagagacattgacagtggtctgaccagttagagagagcataggttcctccgcttagagtgaggctaaggagatagcggcctgatggtggacggtggcattgggaaggcaacgagatgacagtgacgccgtcgtagctacgaggacaatggatagtggttatggcgagagaatcccgatcatgtaattaggtgatggtgagttgctcgagacggaagatgagaatagggtcaggcacctcgctagattggttaggtggtggtggtcatgaattcagcgacaagaccattagagacgaggaagacaacagcgtaaggacataagaaatggtgatggcacatgagaagccatctcaaggaggatgatggcaatagggatggcgatggaggtgcttgtcggcgagcatcaccagcgttgagggagatgccattgaggctaaatggtggtggtgcagagggcatgattggttcagcgtagcacagggtgaggttaggaggagcgatgtggaagtggtggctagggcttgactatttgtttgtattggagggtggagataaaatgtagattttagatgacagatcttgcgattgagaatttgaaaaatctggtgggggattttcttactaatattacttggggttatttggttagttgcttcactttgtaatgccacattttgattatggcgtagttttctaggtaaatgtttgcttcgcccccacacctttttagccttaggacatagtatatgttatactactacctccgtttcaaaatatattttagtccaatttatagtattgggctgagtttagtgtcatagaaaatagggggagaatttactatttgcaactaccaaaatgatagcactcaaataccactctcctaaattttcattcccaaatgccatccgaacgcacatgtcagcctcacttttcattcaaacataagtggtgaggtccgggtgacatttaggaatgaatatttgggagagtggcatttgagcactagtattttggtagagtgacaaatggtaaattcttcaattgagtttaaactatcagtgtttgagtgtagaaaatccttatccgttctcaccatatctggcttattcctcaaataagccaaacggtatatttgcataaaaaataatttatgaataaaacttttatatgtgtgttcttagcgatttgaaaacaaaggctgaaaaaaataaacttcgttaaaaaaatcccaaaaccaaactccaaatttaatttttggctgataagtccaaaatctagtggttacaagagccttagtagcaccttagattctgggttcgcctccccgtggaagcgaattttccaggatttaacggctttgtgctttcagtggtaggcaacgtaaccgtcgacaacgaggcgcccgtggtgactttgtcaatctccaagatggatttgccagcacagtcttcgaagatgctcataggggtagggtttacgtgtatgcgttcataggtgtgaggatgctcataggggtagggtttacgtgcgtgcgttcataagggtgagtatgtgttgtgagtgtttgtgttgtactgtgtaattcttaaagaaaaaaacttcaaatttaaattttggctgataagtccataaacataagcatagtcagcctgctaaccttcttctgtatccatgcggcgtagcgcgtgaaatcttttttagtagctctgaaacaaacaaatgcagccacgagattagggatggcaacggggcggggcagggccaggttggtcaagaatgcccccgcccccgctcccgttccctgactctctcccccgttcccgcccccgaagcccagatcgggtgcaaaacaagacctgcccccgcacaccttccgggtccccgcacccccgggttagtagcggcgccgaccagtaaaatatggttgtttctcctctacaggc

SEQ ID NO: 11 - DNA, 30 bp, Artificial Sequence
  primer
ggtaaattcttcaattgagtttaaactatc

SEQ ID NO: 12 - DNA, 16 bp, Artificial Sequence
  probe
ttctacactcaaacac

SEQ ID NO: 13 - DNA, 25 bp, Artificial Sequence
  primer
agatatggtgagaacggataaggat

SEQ ID NO: 14 - DNA, 73 bp, Artificial Sequence
  primer
ggtaaattcttcaattgagtttaaactatcagtgtttgagtgtagaaaatccttatccgttctcaccatatct

SEQ ID NO: 15 - DNA, 18 bp, Artificial Sequence
  primer
ttgcgcctgaacggatat

SEQ ID NO: 16 - DNA, 19 bp, Artificial Sequence
  probe
tccgagccgtccgtgcgtc

SEQ ID NO: 17 - DNA, 20 bp, Artificial Sequence
  primer
cggttgatcttttcgggatg

SEQ ID NO: 18 - DNA, 81 bp, Artificial Sequence
  primer
ttgcgcctgaacggatatctttcagtttgtaaccaccggatgacgcacggacggctcggatcatcccgaaaagatcaaccg

SEQ ID NO: 19 - DNA, 2178 bp, Artificial Sequence
  DsRed gene
cgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggcgcgccaaccgtctcttcgtgagaataaccgtggcctaaaaataagccgatgaggataaataaaatgtggtggtacagtacttcaagaggtttactcatcaagaggatgcttttccgatgagctctagtagtacatcggacctcacatacctccattgtggtgaaatattttgtgctcatttagtgatgggtaaattttgtttatgtcactctaggttttgacatttcagttttgccactcttaggttttgacaaataatttccattccgcggcaaaagcaaaacaattttattttacttttaccactcttagctttcacaatgtatcacaaatgccactctagaaattctgtttatgccacagaatgtgaaaaaaaacactcacttatttgaagccaaggtgttcatggcatggaaatgtgacataaagtaacgttcgtgtataagaaaaaattgtactcctcgtaacaagagacggaaacatcatgagacaatcgcgtttggaaggctttgcatcacctttggatgatgcgcatgaatggagtcgtctgcttgctagccttcgcctaccgcccactgagtccgggcggcaactaccatcggcgaacgacccagctgacctctaccgaccggacttgaatgcgctaccttcgtcagcgacgatggccgcgtacgctggcgacgtgcccccgcatgcatggcggcacatggcgagctcagaccgtgcgtggctggctacaaatacgtaccccgtgagtgccctagctagaaacttacacctgcaactgcgagagcgagcgtgtgagtgtagccgagtaatggcctcctccgagaacgtgatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgttaagcaccccgccgacatccccgactacaagaagctgtccttccccgagggcttcaagtgggagcgcgtgatgaacttcgaggacggcggcgtggccaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagttcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggcc

SEQ ID NO: 20 - DNA, 4681 bp, Artificial Sequence
  ZmAA gene
agcttgcatgcctgcaggtcgactctagaggatctgcaccggacactgtctggtggcataccagacagtccggtgtgccagatcagggcacccttcggttcctttgctcctttgcttttgaaccctaactttgatcgtttattggtttgtgttgaacctttatgcacctgtggaatatataatctagaacaaactagttagtccaatcatttgtgttgggcattcaaccaccaaaattatttataggaaaaggttaaaccttatttccctttcaatctccccctttttggtgattgatgccaacacaaaccaaagaaaatatataagtgcagaattgaactagtttgcataaggtaagtgcataggttacttagaattaaatcaatttatacttttacttgatatgcatggttgctttcttttattttaacattttggaccacatttgcaccacttgttttgttttttgcaaatctttttggaaattctttttcaaagtcttttgcaaatagtcaaaggtatatgaataagattgtaagaagcattttcaagatttgaaatttctccccctgtttcaaatgcttttcctttgactaaacaaaactccccctgaataaaattctcctcttagctttcaagagggttttaaatagatatcaattggaaatatatttagatgctaattttgaaaatataccaattgaaaatcaacataccaatttgaaattaaacataccaatttaaaaaatttcaaaaagtggtggtgcggtccttttgctttgggcttaatatttctccccctttggcattaatcgccaaaaacggagactttgtgagccatttatactttctccccattggtaaatgaaatatgagtgaaagattataccaaatttggacagtgatgcggagtgacggcgaaggataaacgataccgttagagtggagtggaagccttgtcttcgccgaagactccatttccctttcaatctacgacttagcatagaaatacacttgaaaacacattagtcgtagccacgaaagagatatgatcaaaggtatacaaatgagctatgtgtgtaatgtttcaatcaaagtttcgagaatcaagaatatttagctcattcctaagtttgctaaaggttttatcatctaatggtttggtaaagatatcgactaattgttctttggtgctaacataagcaatctcgatatcacccctttgttggtgatccctcaaaaagtgataccgaatgtctatgtgcttagtgcggctgtgttcaacgggattatccgccatgcagatagcactctcattgtcacataggagagggactttgctcaatttgtagccatagtccctaaggttttgcctcatccaaagtaattgcacacaacaatgtcctgcggcaatatacttggcttcggcggtagaaagagctattgagttttgtttctttgaagtccaagacaccagggatctccctagaaactgacaagtccctgatgtgctcttcctatcaattttacaccctgcccaatcggcatctgaatatcctattaaatcaaaggtggatcccttggggtaccaaagaccaaatttaggagtgtaaactaaatatctcatgattcttttcacggccctaaggtgaacttccttaggatcggcttggaatcttgcacacatgcatatagaaagcatactatctggtcgagatgcacataaatagagtaaagatcctatcatcgaccggtataccttttggtctacggatttacctcccgtgtcgaggtcgagatgcccattagttcccatgggtgtcctgatgggcttggcatccttcattccaaacttgttgagtatgtcttgaatgtactttgtttggctgatgaaggtgccatcttggagttgcttgacttgaaatcctagaaaatatttcaacttccccatcatagacatctcgaatttcggaatcatgatcctactaaactcttcacaagtagatttgttagtagacccaaatataatatcatcaacataaatttggcatacaaacaaaacttttgaaatggttttagtaaagagagtaggatcggctttactgactctgaagccattagtgataagaaaatctcttaggcattcataccatgctgttggggcttgcttgagcccataaagcgcctttgagagtttataaacatggttagggtactcactatcttcaaagccgagaggttgctcaacatagacctattcaccccatttgatcacttttttggtccttcaggatctaatagttatgtataatttagagtctcttgtttaatggccagatatttctaattaatctaagaatttatgatattttttaattttttatcatgtctgatgagaattaacataaaggctcaattgggtcctgaattaataatagagtgaaaattaatccagaggctctattagaaccttcaattagtaataccaagatatatataagatagtagagtatagtttaaatgttggcattgttcattctttcttttgttatttaatttatgctttccacggtggttagtggttacttctgaagggtccaaataatgcatgaagagtttgaggacaagaagtctgccctaaaaatagcgatgcaaaggcatggtgtccaagccatacatatagcgcactaattttatcagcagaacaatggtatttataggtcctagtgcccaggcaacaagagacacgaataaagcatcgatcacgacaccatggcggcgacaatggcagtgacgacgatggtgacgaggagcaaggagagctggtcgtcattgcaggtcccggcggtggcattcccttggaagccacgaggtggcaagaccggcggcctcgagttccctcgccgggcgatgttcgccagcgtcggcctcaacgtgtgcccgggcgtcccggcggggcgcgacccgcgggagcccgatcccaaggtcgtccgggcggcctgcggcctggtccaggcacaagtcctcttccaggggtttaactgggagtcgtgcaagcagcagggaggctggtacaacaggctcaaggcccaggtcgacgacatcgccaaggccggcgtcacgcacgtctggctgcctccaccctcgcactccgtctcgccacaaggctacatgccaggccgcctatacgacctggacgcgtccaagtacggcacggcggcggagctcaagtccctgatagcggcgttccacggcaggggcgtgcagtgcgtggcggacatcgtcatcaaccaccggtgcgcggaaaagaaggacgcgcgcggcgtgtactgcatcttcgagggcgggactcccgacgaccgcctggactggggccccgggatgatctgcagcgacgacacgcagtactcggacgggacggggcaccgcgacacgggcgaggggttcgcggcggcgcccgacatcgaccacctcaacccgcgcgtgcagcgggagctctccgcctggctcaactggctcaggtccgacgccgtggggttcgacggctggcgcctcgacttcgccaagggctactcgccggccgtcgccagaatgtacgtggagagcacggggccgccgagcttcgtcgtcgcggagatatggaactcgctgagctacagcggggacggcaagccggcgcccaaccaggaccagtgccggcaggagctgctggactggacgcgggccgtcggcgggcccgccatggcgttcgacttccccaccaagggcctgctgcaggcgggcgtgcagggggagctgtggcggctgcgcgacagctccggcaacgcggccggcctgatcgggtgggcgcccgagaaggccgtcaccttcgtcgacaaccatgacaccgggtcgacgcagaagctctggccgttcccatcagacaaggtcatgcagggctacgcctacatcctcacccatccaggagtcccctgcattttctacgaccacatgttcgactggaacctgaagcaggagatatccacgctgtctgccatcagggcgcggaacggcatccgcgccgggagcaagctgcggatcctcgtggcggacgcggacgcgtacgtggccgtcgtcgacgagaaggtcatggtgaagatcgggacaaggtacggcgtgagcagcgtggtcccgtcggatttccacccggcggcgcacggcaaggactactgcgtctgggagaaagcgagcctccgcgtcccggcggggcgccacctatagcagctcagattgctcagtcttgtgctgcattgcaaacacagcagcacgacactgcataacgtcttttccttgagatctgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagctactttagactgtgttcggcgttccccctaaatttctccccctatatctcactcacttgtcacatcagcgttctctttcccctatatctccacg

SEQ ID NO: 21 - DNA, 5534 bp, oryza sativa
tcaattgaagaatttaccatttgtcactctaccaaaatactagtgctcaaatgccactctcccaaatattcattcctaaatgtcacccggacctcaccacttatgtttgaatgaaaagtgaggctgacatgtgcgttcggatggcatttgggaatgaaaatttaggagagtggtatttgagtgctatcattttggtagttgcaaatagtaaattctccccctattttctatgacactaaactcagcccaatactataaattggactaaaatatattttgaaacggaggtagtagtataacatatactatgtcctaaggctaaaaaggtgtgggggcgaagcaaacatttacctagaaaactacgccataatcaaaatgtggcattacaaagtgaagcaactaaccaaataaccccaagtaatattagtaagaaaatcccccaccagatttttcaaattctcaatcgcaagatctgtcatctaaaatctacattttatctccaccctccaatacaaacaaatagtcaagccctagccaccacttccacatcgctcctcctaacctcaccctgtgctacgctgaaccaatcatgccctctgcaccaccaccatttagcctcaatggcatctccctcaacgctggtgatgctcgccgacaagcacctccatcgccatccctattgccatcatcctccttgagatggcttctcatgtgccatcaccatttcttatgtccttacgctgttgtcttcctcgtctctaatggtcttgtcgctgaattcatgaccaccaccacctaaccaatctagcgaggtgcctgaccctattctcatcttccgtctcgagcaactcaccatcacctaattacatgatcgggattctctcgccataaccactatccattgtcctcgtagctacgacggcgtcactgtcatctcgttgccttcccaatgccaccgtccaccatcaggccgctatctccttagcctcactctaagcggaggaacctatgctctctctaactggtcagaccactgtcaatgtctctggacataatcaacattgttggagaggcagagagcagaaattgtgaggtagatataaaggttagatatttgtaagattttcctcttaattttcaatcgaatgtggtttagggaaatggatattttttttactagactggcgtatagcaatccgaagacgaagaaagaaggaggaggagtaaaacacaaaaaaagaaaccaaccgtcttaaaacttacaactttaccattgggcaatacaactgcaatgggccaagaagttaatttaaagttaagagcaaattcatttggaccaccttttgttacagatgcttcactttggaccacataccacccatctctcttctcgagcatgaacaatctcgattacattggctcctactcatcaataaactctcacatatatatgtaaaaccattcatcggtatatgacaagttatatatggataaaagagttgaggatgggtaagaataataaccggtataaagtgagaacattgataaacatcgctaataaaagttcgtctatagtaaaatttactctaaaattaaatcacctaatattttaatatttttttgtacaaatggaccgtttcaatggggctttatcagatttagttgagatgcatacatggtaagcaccgtcataatcttgcccaagagctgacccaactcattaaaattacgcttcttttacgacttaataaatcaagaagaaaccattgaaatccagcctgccccgactgtctcgtaacagaaaaataactcagcaacgactaaattatgattttaaaatggcaaaaatatcaaagcacgttcgaaacaatcgcaagattggcaagtaaactctcctgcttgcttgcttacaaccacatcagatcattgatcaatgtttcatcagctcatcacttctgcatgcatgttatattcttctcagggctccacaatttacaaagctgctcgaagatcttctttgcagtgcaaagcaatctgcaagattattcaagacatctactcttgatctaccattgagctaactccggatatataaacagaccgaacgtttcgtcccaggggaatgtgaaagttagcgaatttgcccggcgaaaatggcagcacttggccgcgcgagctcgtcggcgccggtgcttgccgccgccgccgccgtgctcctctcgctctgcctcgccgcgctctcggaagagcaaggtgcgtaaacgttgcgttgtatctttgcgttgatgcgtgttgcgtcgtcgtcgtgttcatggcgtgcgatggcgttgtgcagagcaactggagaacctgcggttcgtgcggcacgcgcaggacgcgccgctggtgtcgagctacaactacatcgtcatcggcggcggcacggcggggtgcccgctggcggcgacgctgtcggagcactcgcgcgtgctgctgctggagcgcggcggcctgccgtacgccaacatgtcgagcgagcagcacttcacggacgcgctggccgacacgtcgccggcgtcgccggcgcagcggttcatctcggaggacggcgtggtgaacgcccgggcgcgggtgctcggcggcgggagctgcctcaacgccgggttctacacgcgggcgagcaacgagtacgtgcgcgcctccgggtgggacgcgcggctggtgaactcgtcgtaccggtgggtggagcgctcgctggtgttccgccccgacgtgccgccgtggcaggcggcgctccgcgacgcgctgctcgaggtcggcgtcacgcccgacaacggcttcaccttcgaccacgtcaccggcaccaagatcggcggcaccatcttcgacaactccggccagcgccacaccgccgccgacttcctccgccacgcccgcccccgcggcctcaccgtcctcctctacgccaccgtctcccgtatcctcttcaaaagccaaggtacacagctacgatgaaaatggaaaatgtgctgtgcgccgaagaagcttgacctcacgacggcgagcttttgccatggcgtgcagacggcgtcccatacccggtggcgtacggggtggtgttctcggacccgctgggggtgcagcaccgggtgtacctccgcgacggcgacaagaacgaggtgatcgtgtcggcggggacgctggggagcccgcagctgctgatgctgagcggcgtcgggccgcaggcgcacctggaggcgcacggcatcgaggtgatcgtggaccaacccatggtcgggcagggcgtcgccgacaacccgatgaactcggtgttcatcccgtcgccggtgccggtggagctctccctggtgcaggtcgtcggcatcacccgctccggcagcttcatcgagggggtgagcgggtcggagttcggcatgccggtgtcggacggcgcgctccggtgggcgcgcagcttcgggatgctgtcgccgcagacggggcagctcggcacgctgccgccgaagcagaggacgccggaggcgctgcagcgggcggcggaggcgatgatgcggctggacaggagggcgttccggggaggcttcatcctggagaagatcctcgggccggtgtcctccggccacgtcgagctgcgaaccaccgacccgagggcgaacccgtcggtgacgttcaactacttccgcgaggcagaggatctggagcggtgcgtccatggcatcgagacgatcgagcgggtgatccagtcgcgggccttctccaacttcacctacgccaacgcctccgtcgagtccatcttcaccgattccgccaacttccccgtcaacctgctgccgcgccatgtcaacgactcgcgctcgccggagcagtactgcatggacaccgtcatgaccatctggcactaccacggcggctgccatgtcggcgccgtcgtcgacgacgattaccgggtgttcggggtgcaggggctcagggtgatcgacagctccaccttcaagtactcccccggcaccaaccctcaggccaccgtcatgatgctcggcaggtaactggcatcattttagctcatgaaagtgcattgccatgagtaacaacacactaacagtatagttttcaatatggacactgggcaggtatatgggtgtgaagattcagtccgagagatggaagaaatgatgaacaaaagataatttcgtttcaggagcaaaaaaatgcatgtaattcaaggaaaagaaaatgttcaactgtctttagagtttagagtagattttatttgcacccacttaatttttactcttctctagacataggttcagtatctgcttgttgattatgtaaccttgaagaagcattgcaaaaacaaagcggaaacttatgttaccaagggcatgacgaagaaataaatggattagatttcattgacacttagaaaatggaaccagcaaatcaaggctgaaaataattacactagaaacttattttaatggctttacatgtcgctacatacttaaatcaatcaaagttgctaccaaagccatgttccctaaacagagggttccgggctttcaaacattcttaatcttctatacattgataaaaagtatacataaaaagaaaacctattaagatggaaatgttgaattctcttaagaaaggcataaaaaatgcagggtaataaccttttcttgtcatgtcctacttggtttcaacctatattgctagcaaaattttcacgctatctataacctcaggaagcagaatagctcagaatcactagcaattgtatacttacagataatcgaaattcatattgtgaggttttcttggatggctcggaatgatccacagatgttaccacccatgtctgggaatgacgtacatcctggcaagggtttcaccttgcccttacggtcaacacttacagggtacttcattaggtggcctctcatctcagtcacctcattatcaacaaatcgttcccagttctgttcaccgatttggcgaactcgtctcatgcattccatggtctctggatagttgaagccttcctcaaccactccaatatgctccgcccaaagagacattctgtacccataaatctacacatgaaatcagcagatttgaattagagtgtggttctagaaatatcagtcatcctaaaataggtatatccctcaaaccaagagacaactagtcgagattccttcacagttgtacagggctatttgaaatatgactgtacttgatatgcaactttttctctatttctacaaatacatttgtattttccatacatttgtagctacaatctggtcccaccatgatggatggtgtagatagctctctttttcttcaattaacatattcttgagaatgaatggtagcttctctttttttcttccaattaacatattcttaagagtgagtggtagcttttttttagccttatctaaaaaaatatgattgatagggtacgaatttccctttaggttactctgccaaaacaaaagataattgcaacaaatatatatggctggatt